SEQUENCE LISTING (10 sequences)

SEQ ID NO: 1 - DNA, 22 bp, Artificial Sequence
  ODN 2395
tcgtcgttttcggcgcgcgccg

SEQ ID NO: 2 - RNA, 10 nt, Artificial Sequence
  TLR13 bacterial ribosomal RNA sequence (unmethylated)
cggaaagacc

SEQ ID NO: 3 - protein, 26 aa, Artificial Sequence
  albumin binding motif
GVSDFYKKLIDKAKTVEGVEALKDAI

SEQ ID NO: 4 - protein, 26 aa, Artificial Sequence
  albumin binding motif
GVSDFYKKLIDKAKTVEGVEALKDEI

SEQ ID NO: 5 - protein, 26 aa, Artificial Sequence
  albumin binding motif
GVSDFYKKLIDKAKTVEGVEALKEAI

SEQ ID NO: 6 - protein, 26 aa, Artificial Sequence
  albumin binding motif
GVSDFYKKLIDKAKTVEGVEALKEEI

SEQ ID NO: 7 - protein, 26 aa, Artificial Sequence
  albumin binding motif
GVSDFYKKLIEKAKTVEGVEALKDAI

SEQ ID NO: 8 - protein, 26 aa, Artificial Sequence
  albumin binding motif
GVSDFYKKLIEKAKTVEGVEALKDEI

SEQ ID NO: 9 - protein, 26 aa, Artificial Sequence
  albumin binding motif
GVSDFYKKLIEKAKTVEGVEALKEAI

SEQ ID NO: 10 - protein, 26 aa, Artificial Sequence
  albumin binding motif
GVSDFYKKLIEKAKTVEGVEALKEEI

(19) United States
(12) Patent Application Publication
(10) Pub. No.: US 2023/0405129 A1
(43) Pub. Date: Dec. 21, 2023

(54) PROINFLAMMATORY PRODRUGS

(71) Applicant: THE BOAD OF REGENTS OF THE UNIVERSITY OF TAXAS SYSTEM, Austin, TX (US)

(72) Inventors: Hans HAMMERS, Austin, TX (US); Xiankai SUN, Coppell, TX (US); Sashi DEBNATH, Dallas, TX (US)

(73) Assignee: THE BOAD OF REGENTS OF THE UNIVERSITY OF TAXAS SYSTEM, Austin, TX (US)

(21) Appl. No.: 18/032,760

(22) PCT Filed: Oct. 20, 2021
(86) PCT No.: PCT/US2021/055885

§ 371 Date: Apr. 19, 2023

Related U.S. Application Data
(60) Provisional application No. 63/093,964, filed on Oct. 20, 2020.

(51) Int. Cl. A61K 47/54; A61K 47/65; A61K 47/60; A61K 51/04; A61K 51/08; A61K 45/06; A61P 35/00

(52) U.S. Cl. CPC A61K 47/542; A61K 47/65; A61K 47/60; A61K 47/545; A61K 51/0497; A61K 51/0402; A61K 51/0461; A61K 51/088; A61K 45/06; A61P 35/00; A61B 6/4057

(57) ABSTRACT

Provided herein are prodrugs comprising targeting moieties that specifically bind extracellular antigens, enzyme-cleavable linkers, and innate immune system activators. An enzyme-cleavable linker can covalently link a targeting moiety to an innate immune system activator. Also provided are methods of treating cancer, methods of imaging cancer, and methods of monitoring treatment of cancer.

### BACKGROUND

More than 65,000 new cases of kidney cancer occur annually, leading to about 15,000 deaths in the United States, with about ten times this number worldwide. Although frequent use of imaging studies has led to earlier detection, as many as 30% of patients progress with metastatic disease after nephrectomy. Kidney cancer is a challenging disease that has been notoriously difficult to treat. Generally resistant to classic chemotherapy, the main treatment option for kidney cancer has been drugs which target the vascular endothelial growth factor (VEGF) pathway, thus suppressing new blood vessel formation and with it the tumor's unfettered access to oxygen and nutrients. This treatment approach has been the standard of care for treatment-naïve patients for more than a decade.

A significant advance in cancer therapy is immunotherapy. Particularly impactful because of their broad application are immune checkpoint inhibitors, i.e., antibodies targeting the CTLA or PD1/PDL1 pathway, which take the brakes off of the immune response to promote an antitumor effect. However, even in cancers traditionally regarded as immune responsive, such as melanoma or renal cell carcinoma, many patients fail to respond. The inflammatory state of tumors before starting immunotherapy is associated with a better and deeper response. Specifically, lack of a priori immune recognition of “cold” tumors is a primary reason for failure of cancer immunotherapy. Thus, there exists a need for improved immunotherapies that include broad activation of inflammation in tumors.

### SUMMARY

A prodrug comprising a targeting moiety that specifically binds to an extracellular antigen; a first enzyme-cleavable linker; and a first innate immune system activator, wherein the first linker covalently links the targeting moiety to the innate immune system activator is provided. The extracellular antigen can be an extracellular tumor antigen.

The extracellular antigen can be a prostate-specific membrane antigen (PSMA), carbonic anhydrase 9 (CA9), or fibroblast activation protein (FAP). The targeting moiety can be a molecule that binds specifically to PSMA, CA9, or FAPI. The first enzyme-cleavable linker can be a cathepsin-cleavable linker or a legumain-cleavable linker. The enzyme-cleavable linker can be an azide-polyethylene glycol (PEG)-valine-citrulline-p-aminobenzyl (PAB)-p-nitrophenol (PNP) linker or an alanine-alanine-asparagine legumain linker. The prodrug can further comprise a spacer linker between the targeting moiety and the first linker. The linker spacer can be an azide-PEG-maleimide spacer linker or an N-hydroxysuccinimide (NHS)-PEG-Alkyne spacer linker. The prodrug can further comprise an albumin binding moiety. The first innate immune system activator can comprise a Toll-like Receptor (TLR) agonist or a Stimulator of Interferon Genes (STING) agonist. The TLR agonist can be a TLR7 agonist, a TLR8 agonist, a TLR9 agonist, or a mixed TLR7/8 agonist. The TLR7 agonist can be gardiquimod, imiquimod, telratolimod, or their analogs. The mixed TLR7/8 agonist can be resiquimod and its analogs. The TLR9 agonist can be ODN-2395, CMP-001, or MGN1703. The STING agonist can be a cyclic dinucleotide. The prodrug can further comprise a radiolabel moiety (via a prosthetic group or a chelator), a second activator of the innate immune system, or a combination thereof. The prosthetic group can comprise a moiety for labeling with a radionuclide selected from ¹⁸F, ¹²³I, ¹²⁴I, ¹³¹I, ⁷⁵Br or ⁷⁶Br linked to the prodrug by a second linker. The chelator can be a bifunctional chelator, such as 1,4,7,10-tetraazacyclodecane-1,4,7,10-tetraacetic acid (DOTA), for labeling with a radionuclide for imaging selected from ⁶⁷Ga, ⁶⁸Ga, ⁶⁰Cu, ⁶¹Cu, ⁶²Cu, ⁶⁴Cu, ⁸⁹Zr, ¹¹¹In, ¹⁷⁷Lu, and ^{99m}Tc and/or a radionuclide for radiation therapy selected from ⁶⁷Cu, ¹⁷⁷Lu, ⁹⁰Y, ²¹¹At, ²²⁵Ac, and ²²⁷Th. The second innate immune activator can comprise a proinflammatory molecule linked to the prodrug by a third linker.

A prodrug selected from prodrugs (1) to (15) is provided.

A method of treating cancer in a subject comprising administering to the subject an effective amount of one of the prodrugs described herein is provided.

The cancer can be kidney cancer, renal cancer, prostate cancer, lung cancer, colon cancer, rectal cancer, urinary bladder cancer, melanoma, oral cavity cancer, pharynx cancer, pancreatic cancer, uterine cancer, thyroid cancer, skin cancer, head and neck cancer, cervical cancer, ovarian cancer, breast cancer, or hematopoietic cancer. An immunotherapy can further be administered prior to, simultaneously with, or following administration of the prodrug. For example, a prodrug can be administered about 1, 30, 60, 90 minutes or more after or before administration of immunotherapy. In another example, a prodrug can be administered about 2, 5, 12, 24, 36, 48 hours or more after or before administration of immunotherapy. In another example, a prodrug can be administered about 3, 5, 7, 14, 21, 27 days or more after or before administration of immunotherapy. The immunotherapy can be an interleukin, a cytokine, a chemokine, an immunomodulatory imide drug, CAR-T cells, TCR therapy, a monoclonal antibody, a cancer vaccine, a checkpoint inhibitor, or combinations thereof.

A method of imaging cancer in a subject comprising administering to the subject one of the prodrugs described herein; optionally administering to the subject after, simultaneously with, or before administration of the prodrug; and performing a functional imaging on the subject is provided.

A method of monitoring treatment of cancer in a subject comprising administering to the subject one of the prodrugs described herein; optionally administering to the subject immunotherapy after, simultaneously with, or before administration of the prodrug; and performing a functional imaging on the subject. For example, a prodrug can be administered about 1, 30, 60, 90 minutes or more after or before administration of immunotherapy. In another example, a prodrug can be administered about 2, 5, 12, 24, 36, 48 hours or more after or before administration of immunotherapy. In another example, a prodrug can be administered about 3, 5, 7, 14, 21, 27 days or more after or before administration of immunotherapy. The functional imaging can be positron emission tomography (PET) or single-photon emission computed tomography (SPECT).

### BRIEF DESCRIPTION OF THE DRAWINGS

FIG. 1 shows the PSMA-TLR7 prodrug. The binding moiety is the class PSMA ligand. Gardiquimod is attached to a cathepsin B cleavable linker system, allowing for traceless release.

FIG. 2 shows release of TLR7 activity of the Gardiquimod PSMA prdrug. A PSMA expressing prostate cancer cell line (LnCAP) was used. Untreated LnCAP cells and the PSMA negative mouse kidney cancer cell line RENCA served as negative controls. Cells were exposed to prodrug and then free gardiquimod was eluted from cells and transferred to a reporter cell line. Results demonstrate optical densitometry results and were normalized over control.

FIG. 3 shows TLR7 reporter activity of PSMA negative and PSMA positive reporter cells upon incubation with PSMA11-TLR7 prodrug. LEFT: The normally PSMA negative HEK293 Blue TLR7 Reporter cell line was transduced with a PSMA gene containing lentivirus. Roughly 5% of the transduced cells expressed the gene. RIGHT: Cells were exposed to the PSMA11-TLR7 prodrug. There was no TLR7 reporter activity in the PSMA negative cell line. The partially transduced PSMA positive cell line demonstrates TLR7 activity in a subset of cells roughly reflecting the PSMA positive fraction.

FIG. 4 shows the schematic induction of inflammation in response to proinflammatory TLR prodrugs, which interact with their drug target intracellularly in the lysosome.

FIG. 5 shows the modular design of prodrug building blocks for “click-synthesis.”

FIG. 6 shows a sample synthesis (top) and the structures (bottom) of the indicated TLR prodrugs.

FIGS. 7A-7F show structures of examples of TLR prodrugs. FIG. 7A shows the structures of the indicated TLR7 and TLR9 prodrugs. FIG. 7B shows structures of TLR7 and TLR9 conjugates with FAPI04. FIG. 7C shows structures of albumin-binding motif (ABM) modified TLR7 and TLR9 conjugates with FAPI04. FIG. 7D shows a structure of an irreversible PSMA targeting molecule. FIG. 7E shows structures of a general tri-functional theranostic conjugate design. FIG. 7F shows a structure of CA9-PEG₃-TLR7.

FIG. 8 shows quantitative organ uptake expressed as the percentage of injected dose per gram tissue (% ID/g) derived from the PET imaging data. Mouse PET imaging was performed using a Siemens Inveon PET/CT Multimodality System. Each mouse was sedated with 2% isoflurane anesthesia throughout the scan. Static PET scans were conducted at 1 h p.i. for 15 min and the CT data acquisition was executed at 80 kV and 500 μA with a focal spot of 58 μm. All the PET and CT data were reconstructed and regions of interest (ROIs) were marked as displayed by CT to quantify the tracer uptake as percent injected dose per gram of tissue (% ID/g).

FIG. 9 shows the electrospray ionization mass spectrometry results for the synthesized PSMA-Cathepsin-Gardiquimode prodrug. These results indicate that the synthesized prodrug is as expected.

FIG. 10 shows the liquid chromatography mass spectrometry results for synthesized prodrug PSMA-C5-Cathepsin-Gardiquimode.

FIG. 11 shows the liquid chromatography mass spectrometry results for synthesized prodrug PSMA-Legu-Gardiquimode.

FIG. 12 shows the electrospray ionization spectrometry results for synthesized prodrug PSMA617-Legu-Gardiquimode.

FIG. 13 shows the mass spectrometry results for the synthesized CA9-PEG₃-TLR7 conjugate.

FIG. 14A shows the small animal PET/CT imaging studies evaluated by 15 min PET scan followed by 7 min CT scan. The results confirm that PSMA positive PC3-PIP tumor was clearly observable while the PSMA negative PC3-Flu tumor has very weak image contrast (similar to muscle uptake).

FIG. 14B shows a quantitative uptake analysis, which confirms that conjugate TLR-LEGU-PSMA-[¹⁸F]SFB has higher uptake in PC3-PIP (1.9±0.5% ID/g) tumor than in PC3-Flu (0.8±0.3% ID/g) tumors (n=3).

FIG. 15 shows in vitro testing of CA9 targeted prodrug. At the 10 uM dose, the sample at about 1.3 optical density is CA9-Gardiquimod, the sample at about 0.9 optical density is CA9+Gardiquimod, the sample at about −0.05 optical density is CA9+CA9-Cath-TLR7, and the sample at about −0.1 optical density is CA9-CA9-Cath-TLR7.

FIG. 16 shows the synthesis of proinflammatory prodrug PSMA-Cathepsin-Gardiquimode and the synthesis of proinflammatory prodrug 3.

FIG. 17A shows the synthesis of proinflammatory prodrugs Propergyl-05-PSMA and PSMA-05-Cathepsin-Gardiquimode (2).

FIG. 17B shows a bar graph in which PSMA prodrugs induce TLR7 reporter activity in PSMA positive HEK-Blue cells but not in a PSMA negative reporter cell line when compared to various doses of free gardiquimod. The Y-axis represents optical density.

FIG. 17C shows a line graph in which PSMA prodrugs induce TLR7 reporter activity in PSMA positive HEK-Blue cells but not in a PSMA negative reporter cell line when compared to various doses of free gardiquimod. The Y-axis represents optical density.

FIG. 18 shows the synthesis of proinflammatory prodrug Legumain-Gardiquimode.

FIG. 19 shows the synthesis of proinflammatory prodrugs PSMA-Legu-Gardiquimode (3) and PSMA617-prop.

FIG. 20 shows the synthesis of proinflammatory prodrug CA9-PEG₃-TLR7.

FIG. 21 shows the synthesis of Azido-PEG4-Ala-Ala-Asn(Trt)-PAB-Gardiquimod (prodrug B) and precursor molecule TLR-LEGU-PSMA, standard prodrug TLR-LEGU-PSMA-[¹⁹F]SFB (5a).

### DETAILED DESCRIPTION

The present disclosure provides prodrugs that include small drug conjugates that are useful for the systemic delivery of proinflammatory prodrugs. As used herein, the term “prodrug” means a biologically inactive compound which can be metabolized in the body to produce a biologically active prodrug. Proinflammatory prodrugs can be selectively activated in target cells such as cancer cells and enhance immunotherapy across the whole tumor bulk.

Prodrugs

Proinflammatory prodrugs for the treatment of cancer are provided. The prodrugs can broadly activate innate immune responses in tumors themselves. Activation of innate immune responses in tumors can lead to adaptive, T-cell mediated immune responses not only against mutated proteins, but also against non-mutated but overexpressed tumor-associated antigens and endogenous retroviruses, for example. The compounds provided herein are compounds comprising several components associated with one another.

Prodrugs provided herein can comprise, for example, (i) a targeting moiety that specifically binds to an extracellular antigen such as an extracellular tumor antigen; (ii) an enzyme-cleavable linker; and (iii) an innate immune system activator, wherein the first enzyme-cleavable linker covalently links the targeting moiety to the innate immune system activator.

Targeting Moieties

Prodrugs provided herein can include a targeting moiety that specifically binds to an extracellular antigen such as an extracellular tumor antigen. An extracellular tumor antigen can be any molecule expressed on the surface of a cancer cell, such as a transmembrane receptor or transmembrane protein, for example, or any molecule expressed at the surface of a cell present in the tumor microenvironment, such as a stromal cell (e.g., a carcinoma associated fibroblast, CAF), an immune cell (e.g., tumor-associated macrophage, neutrophil, tumor infiltrating lymphocyte, or T cell), a pericyte, or an endothelial cell. By specific binding, it is meant that the targeting moiety can bind to its target extracellular tumor antigen with higher specificity and sensitivity than it would to a non-target antigen (e.g., an extracellular antigen not expressed at the surface of a tumor cell or not expressed at the surface of a cell present in the tumor microenvironment).

A targeting moiety can bind to one or more extracellular antigens (i.e., cell surface proteins), which are proteins or polypeptides present on a cell surface. In an embodiment, an extracellular antigen is a tumor antigen (i.e., specific tumor proteins) or other, normally occurring surface proteins, like PSMA on endothelial cells or FAP on fibroblasts. A targeting moiety can bind to one or more extracellular antigens such as extracellular tumor antigens. An extracellular antigen such as an extracellular tumor antigen can have more than one binding site for a targeting moiety.

In some embodiments, a targeting moiety of a prodrug can specifically bind to an extracellular antigen such as an extracellular tumor antigen. An extracellular tumor antigen can be a tumor-specific antigen (TSA) that is present only on tumor cells or a tumor-associated antigen (TAA) that is present on some tumor cells and some normal cells. Exemplary extracellular tumor antigens include products of mutated oncogenes, products of mutated tumor suppressor genes, products of mutated genes other than oncogenes or tumor suppressors, tumor antigens produced by oncogenic viruses, altered cell surface glycolipids and glycoproteins, oncofetal antigens, and others. An extracellular tumor antigen can also be a molecule that is more highly expressed on a tumor cell than a normal cell. An extracellular tumor antigen can also be expressed on non-tumor cells, such as the tumor-associated neovasculature, for example. An extracellular tumor antigen can be specifically expressed in a particular type of cancer or be expressed in many different types of cancer.

Exemplary extracellular tumor antigens include Prostate-Specific Membrane Antigen (PSMA), carbonic anhydrase 9 (CA9), integrins (e.g., αᵥβ₃), alphafetoprotein (AFP), carcinoembryonic antigen (CEA), CA-125, MUC-1, epithelial tumor antigen (ETA), tyrosinase, melanoma-associated antigen (MAGE), abnormal products of ras and p53, octreotide receptor and others. In some embodiments, the extracellular tumor antigen is PSMA. In some embodiments, the extracellular tumor antigen is CA9. In some embodiments, the extracellular tumor antigen is fibroblast activation protein (FAP). In some embodiments, the extracellular tumor antigen is PSMA and the targeting moiety is PSMA-11 or PSMA-617. In some embodiments, the extracellular tumor antigen is CA9 and the targeting moiety is CA9 small molecule binder. In some embodiments, the extracellular tumor antigen is FAP and the targeting moiety is a FAP inhibitor, such as FAPI-04. In some embodiments, the targeting moiety is PSMA-11, PSMA-617, a CA9 small molecule binder, or FAPI-04.

Enzyme-Cleavable Linkers

A prodrug can comprise one enzyme-cleavable linker. Methods for attaching two individual elements can require the use of a linker. The term “linker” as used herein refers any bond, small molecule, or other vehicle that allows an innate immune system activator to be delivered at a targeted area, tissue, or cell, for example, by physically linking an innate immune system activator to a targeting moiety. A linker can be any chemical moiety that is capable of linking compounds. Linkers can be susceptible to acid-induced cleavage, light-induced cleavage, peptidase-induced cleavage, esterase-induced cleavage and disulfide bond cleavage at conditions under which the prodrug remains active. Linkers are classified upon their chemical motifs, including disulfide groups, hydrazine or peptides (cleavable), or thioester groups (non-cleavable). Linkers also include charged linkers, and hydrophilic forms thereof.

The term “cleavable linker” as used herein, refers to linker moieties that are capable of degrading under various conditions. Conditions suitable for cleavage can include, but are not limited to pH, UV irradiation, enzymatic activity, temperature, hydrolysis, elimination, and substitution reactions, and thermodynamic properties of the linkage. Any enzyme-cleavable linker can be used including, for example, peptide or non-peptide linkers.

Peptide linkers can be, for example, valine-citrulline (Val-Cit) containing linkers and phenylalanine-lysine (Phe-Lys) containing linkers. Val-Cit and Phe-Lys linkers can be cleaved by cathepsin B, for example. Additional peptide linkers include, for example, glycine-phenylalanine-leucine-glycine (Gly-Phe-Leu-Gly) linkers and alanine-leucine-alanine-leucine (Ala-Leu-Ala-Leu) linkers. Peptide linkers can be cleaved by lysosomal and endosomal enzymes, such as cathepsin B and legumain, for example.

Non-peptide linkers include, for example, azide-PEG-maleimide linkers, β-glucoronide based linkers, or self-immolative linkers. β-glucoronic-acid based linkers can be cleaved by β-glucoronidase or β-glucoronic-acid. Self-immolative linkers include photolabile O-nitrobenzyl group incorporated linkers that can be cleaved under light irradiation. Azide-PEG-maleimide linkers can be cleaved by endosomal enzymes such as cathepsin B, for example. Non-peptide linkers can be cleavable or non-cleavable.

An enzyme-cleavable linker can influence release of the innate immune system activator from the prodrug compound. Cleavage rate and drug release rate can be expressed as half-life of a drug conjugate. For example, a prodrug can comprise a cleavage linker having a half-life of about 1 min, about 5 min, about 10 min, about 20 min, about 25 min, about 30 min, about 35 min, about 40 min, about 45 min, about 50 min, 55 min, about 1 hour, about 2 hours, about 3 hours, about 6 hours, about 7 hours, about 8 hours, about 9 hours, about 10 hours, about 11 hours, about 12 hours, about 18 hours, about 24 hours, about 36 hours, about 48 hours, about 3 days, about 4 days, about 5 days, about 6 days, about 10 days, about 11 days, about 12 days, about 13 days, about 14 days, about 15 days, about 16 days, about 17 days, about 18 days, about 19 days, about 20 days, about 21 days, about 23 days, about 24 days, about 25 days, about 26 days, about 27 days, about 28 days, about 29 days, about 30 days, and any number or range in between.

An exemplary cleavage linker is a cathepsin B cleavable linker. Additional cleavage linkers include glycine-phenylalanine-leucine-glycine (Gly-Phe-Leu-Gly) linkers and alanine-leucine-alanine-leucine (Ala-Leu-Ala-Leu) linkers. An exemplary cleavage linker is also a legumain cleavable linker.

In some embodiments, the first enzyme-cleavable linker is an azide-PEG-valine-citrulline-p-aminobenzyl (PAB)-p-nitrophenol (PNP) linker or an alanine-alanine-asparagine legumain linker.

An enzyme-cleavable linker can covalently link a targeting moiety to an innate immune system activator. Covalent linkage of a targeting moiety and an innate immune system activator can be direct linkage, i.e., linkage that does not include other moieties such as spacers, for example, between the targeting moiety and the innate immune system activator. Covalent linkage of a targeting moiety and an innate immune system activator can be an indirect linkage, i.e., linkage that includes other moieties such as spacers, for example, between the targeting moiety and the innate immune system activator.

Spacer Linker

A prodrug can comprise one or more spacer linkers. As used herein, “spacer” and “spacer linker” are used interchangeably, and refer to linkers that can be used to maintain a conjugate's flexibility for effective binding to the target. Spacers can be included to separate the innate immune system activator component of the prodrug from other components of the prodrug, such as a targeting moiety. A spacer can help to avoid steric hindrance and crowding and to control the site and rate of release of an innate immune system activator from the prodrug.

In some embodiments, the prodrug can comprise a spacer linker between the targeting moiety and the first linker. For example, an azide-PEG-valine-citrulline-p-aminobenzyl (PAB)-p-nitrophenol (PNP) linker includes a self-immolative p-aminobenzyl alcohol group (PAB) spacer. Effects on drug structure and cytotoxic activity can be avoided by using a self-immolative spacer. As another example, an azide-PEG-maleimide linker includes PEG spacers. Spacers such as PEG, for example, can be hydrophilic and provide for greater solubility.

Prodrugs can comprise 2, 3, 4, 5 or more spacers. In some embodiments, a prodrug can comprise a spacer between the targeting moiety and a linker. In some embodiments, a prodrug can comprise a spacer between a targeting moiety and a linker, wherein the linker is a PEG. In some embodiments, a prodrug can comprise a spacer between a targeting moiety and a TLR agonist. In some embodiments, a prodrug can comprise a spacer between a linker and a TLR agonist.

In some embodiments, the linker spacer can be an azide-PEG-maleimide spacer linker or an N-hydroxysuccinimide (NHS)-PEG-Alkyne spacer linker.

Albumin Binding Motif

Many gram-positive bacteria express surface proteins with the ability to bind serum proteins, for example comprising one or more albumin binding domains or motifs. Albumin is the most abundant protein in plasma. Albumin has an extraordinarily long circulatory half-life of 19 days in humans due to its size, which is above the renal filtration cutoff, and pH-dependent binding to the neonatal Fc-receptor (FcRn). As a consequence, non-covalent association to albumin can be used to extend the half-life of drugs, and therefore to extend their pharmacokinetics properties.

Albumin binding motifs (ABM) can be found in albumin binding proteins or in albumin binding moieties. Examples of albumin binding motifs include, for example, 3-helix bundle serum albumin binding domain (ABD), a consensus albumin binding domain (ABDCon), or an albumin binding FN3 domain. An albumin binding motif can be, for example, a bacterial albumin binding domain, such as a streptococcal protein G domain (Konig & Skerra, (1998) J. Immunol. Methods 218, 73-83) or those as described in US patent application 2003/0069395 or Dennis et al. (2002) J. Biol. Chem. 277, 35035-35043. Other examples of albumin binding motifs can comprise amino acid sequences as described in WIPO patent application WO2014/048977, including, for example:

| (SEQ ID NO: 3) | |
|---|---|
| | GVSDFYKKLIDKAKTVEGVEALKDAI; |
| (SEQ ID NO: 4) | |
| | GVSDFYKKLIDKAKTVEGVEALKDEI; |
| (SEQ ID NO: 5) | |
| | GVSDFYKKLIDKAKTVEGVEALKEAI; |
| (SEQ ID NO: 6) | |
| | GVSDFYKKLIDKAKTVEGVEALKEEI; |
| (SEQ ID NO: 7) | |
| | GVSDFYKKLIEKAKTVEGVEALKDAI; |
| (SEQ ID NO: 8) | |
| | GVSDFYKKLIEKAKTVEGVEALKDEI; |
| (SEQ ID NO: 9) | |
| | GVSDFYKKLIEKAKTVEGVEALKEAI; |
| | and |
| (SEQ ID NO: 10) | |
| | GVSDFYKKLIEKAKTVEGVEALKEEI. |

There are many different types of albumin-binding proteins with different sizes and functions. For example, more than 40 albumin-binding domains have been found in one protein, forming a rod-like structure in a giant cell wall-associated fibronectin-binding molecule. This protein was found on the surface of Staphylococcus aureus and is called Ebh (ECM-binding protein homologue, Uniprot Q2FYJ6). An albumin-binding domain can be a small, three-helical protein domain as found in various surface proteins expressed by gram-positive bacteria. Other albumin binding motifs are described in, for example, US Pat. Publ. 2019/0031727 and US Pat. Publ. 2015/0225464.

An ABM can be located between a targeting moiety and a first innate immune activator. An ABM can be located directly between a targeting moiety and a first innate immune activator, or between a targeting moiety and a linker spacer, between a targeting moiety and a first linker, between a linker and a first innate immune activator, or between a spacer linker and a first innate immune activator.

Innate Immune System Activators

A prodrug can comprise one or more (e.g., 1, 2, 3, 4, 5, or more) innate immune activators. Any innate immune activator can be used, including, for example, Toll-Like Receptor (TLR) agonists and Stimulator of Interferon Genes (STING) agonists, a combination of two or more TLR agonists or a mix of one or more STING agonists and one or moreTLR agonists. As an example, any innate immune activator with an aliphatic amine group for linkage, such as a primary or secondary aliphatic amine, can be used, although innate immune activators with other reactive groups can be used as well.

TLRs and TLR Agonists

Some TLRs can be located on the plasma membrane while others, like TLR7 and TLR9 can be located inside endosomes, where they recognize nucleic acids and nucleotides of intracellular pathogens, for example. Targeting TLR pathways can induce type I interferon and inflammatory responses as a strategy for both antiviral and antitumor therapy. Importantly, TLR pathway activation can lead to dendritic cell maturation thus enhancing antigen processing/presentation and ultimately leading to the activation of antigen-specific T cells.

Agonists of any mammalian TLR can be used as an innate immune system activator, including ligands for TLR1, TLR2, TLR3, TLR4, TLR5, TLR6, TLR7, TLR8, TLR9, TLR10, TLR11, TLR12, and TLR13. Exemplary human TLRs include TLR1, TLR2, TLR3, TLR4, TLR5, TLR6, TLR7, TLR8, TLR9, and TLR10. Exemplary TLR ligands and exemplary TLR expression are shown in Table 1.

TABLE 1
Exemplary TLR Ligands and TLR Expression

| TLR | Exemplary Agonists | Exemplary Expression |
|---|---|---|
| TLR1 | triacyl lipopeptides | monocytes/macrophages, |
| | | some dendritic cells, B |
| | | lymphocytes |
| TLR2 | glycolipids, lipopeptides and | monocytes/macrophages, |
| | proteolipids, lipoteichoic acid, | neutrophils, myeloid |
| | HS70, zymosan (beta-glucan), | dendritic cells, mast cells |
| | others | |
| TLR3 | double-stranded RNA, poly I:C | dendritic cells, B |
| | | lymphocytes |
| TLR4 | lipopolysaccharides (LPS), | monocytes/macrophages, |
| | some heat shock proteins, | neutrophils, myeloid |
| | fibrinogen, heparin sulfate | dendritic cells, mast cells, B |
| | fragments, hyaluronic acid | lymphocytes, intestinal |
| | fragments, nickel, some opoid | epithelium, breast cancer |
| | drugs | cells |
| TLR5 | bacterial flagellin, profilin | monocytes/macrophages, |
| | | some dendritic cells, |
| | | intestinal epithelium, |
| | | breast cancer cells |
| TLR6 | diacyl lipopeptides | monocytes/macrophages, |
| | | mast cells, B lymphocytes |
| TLR7 | imiquimod, resiquimod, | monocytes/macrophages, |
| | gardiquimod, telratolimod, | neutrophils, plasmacytoid |
| | imidazoquinoline (e.g., | dendritic cells, NK cells, |
| | MEI9197, 3M-052), loxoribine, | B lymphocytes |
| | bropirimine, single-stranded | |
| | RNA | |
| TLR8 | small synthetic compounds, | monocytes/macrophages, |
| | single-stranded viral RNA, | some dendritic cells, mast |
| | phagocytized bacterial RNA | cells, some intestinal |
| | | epithelial cells |
| TLR9 | unmethylated CpG | monocytes/macrophages, |
| | oligodeoxynucleotide DNA, | neutrophils, plasmacytoid |
| | e.g., ODN-2395, CMP-001, | dendritic cells, NK cells, |
| | MGN1703, tilsotolimod (e.g., | B lymphocytes, kidney |
| | IMO-2125) | cancer cells, tumor |
| | | endothelial cells |
| TLR10 | triacylated lipopeptides | B cells, intestinal epithelial |
| | | cells, monocytes/macrophages |
| TLR11 | profilin | monocytes/macrophages, |
| | | liver cells, kidney, urinary |
| | | bladder epithelium |
| TLR12 | profilin | neurons, plasmacytoid |
| | | dendritic cells, conventional |
| | | dendritic cells, macrophages |
| TLR13 | bacterial ribosomal RNA | monocytes/macrophages, |
| | sequence “CGGAAAGACC” | conventional dendritic cells |
| | (SEQ ID NO: 2) (unmethylated) | |

Small synthetic TLR7 agonists such as imiquimod (also called Aldara or R-837) and gardiquimod can be used as innate immune system activators. Gardiquimod is a potent TLR7 analog of imiquimod with improved solubility and potency. Importantly, gardiquimod has a reactive secondary aliphatic amine group which allows for chemical conjugation and prodrug release strategies. TLR7 is primarily expressed in the cells of the innate immune system which are virtually always present in solid tumors including renal cell carcinoma and other cancers. Small molecule TLR7 agonists delivered into a tumor microenvironment can diffuse into bystander cell thus causing a proinflammatory field effect.

TLR9 senses an unmethylated CpG motif in bacterial DNA in endosomes. Like TLR7 it is expressed in all activated cellular components of the innate immune response. Additionally, TLR9 is also expressed in the vast majority of kidney cancer cells and other cancer cells as well as endothelial cells. Classic TLR9 agonists are small nucleotides with a phosphorothioate backbone to prevent hydrolysis. After injection into tumors these nucleotides can be endocytosed, bind to their receptor in endosomes and then lead to type I interferon production and inflammation, thus reprogramming the tumor microenvironment. This can provide for the targeted delivery of TLR9 agonists directly to kidney cancer cells and other cancer cells as well as the tumor vasculature. TLR9 agonists are not small molecules, but rather CpG oligonucleotides. Unlike gardiquimod, which can cross membranes by diffusion, TLR9 agonist are less likely to diffuse. Therefore, TLR7/8 can induce more field effect, and TLR9 activation is more likely dependent on target cells.

A prodrug can comprise one or more TLR agonists as innate immune system activators. In some embodiments, a TLR agonist is a TLR7 agonist, a TLR8 agonist, a TLR9 agonist, or a mixed TLR7/8 agonist. In some embodiments, a TLR7 agonist is gardiquimod, imiquimod, or telratolimod. In some embodiments, a TLR9 agonist is ODN-2395, CMP-001, or MGN1703. In some embodiments, the mixed TLR7/8 agonist can be resiquimod.

STING and STING Agonists

A prodrug can comprise one or more (e.g., 1, 2, 3, 4, 5, or more) Stimulator of Interferon Genes (STING) agonists as innate immune system activators. STING is an endoplasmic reticulum (ER) protein encoded by the TMEM173 gene and functions as a cytosolic DNA sensor and an adaptor protein in type I interferon signaling. STING can activate the transcription factors STAT6 and IRF-3 through TBK1, for example, establishing an antiviral response and an innate immune response to intracellular pathogens. STING is expressed in hematopoietic cells in peripheral lymphoid tissues, including T lymphocytes, NK cells, myeloid cells, and monocytes, for example. STING is also highly expressed in lung, ovary, heart, smooth muscle, retina, bone marrow, and vagina, for example.

STING ligands include cyclic dinucleotides and the xanthenone derivative DMXXA. Exemplary cyclic dinucleotides include cGAMP, 2′3′-cGAMP, 3′3′-cGAMP, 2′2′-cGAMP, c-diAMP, c-di-GMP, ADU-S100 (MIW185), MK-1454, BMS-986301, SB 11285, TAK-676, MAVU-104, exoSTING, CRD5500, and others. In some embodiments, a STING ligand is a cyclic dinucleotide. In some embodiments, a cyclic dinucleotide is cGAMP, 2′3′-cGAMP, 3′3′-cGAMP, 2′2′-cGAMP, c-diAMP, c-di-GMP, ADU-S100 (MIW185), MK-1454, BMS-986301, SB 11285, TAK-676, MAVU-104, exoSTING, or CRD5500.

Exemplary Prodrugs

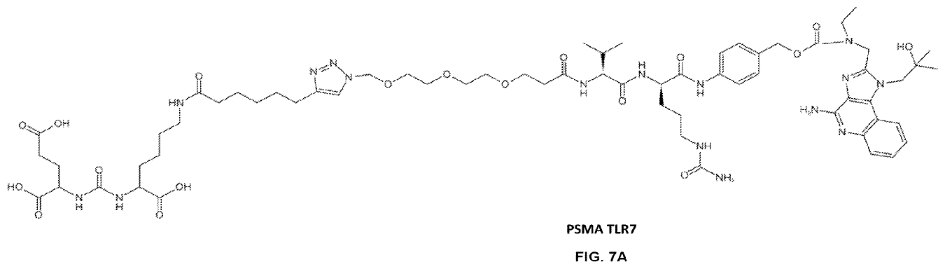

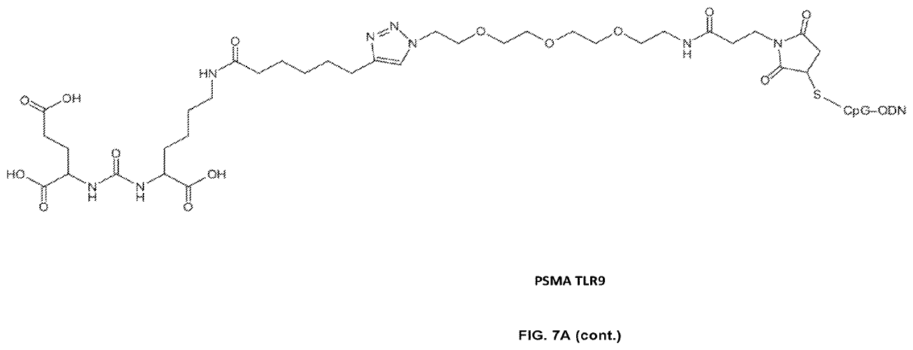

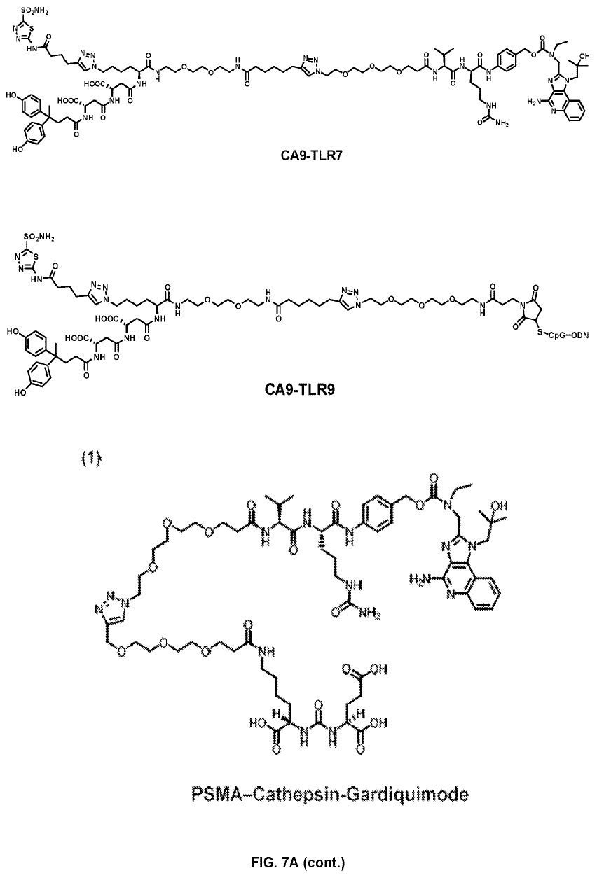

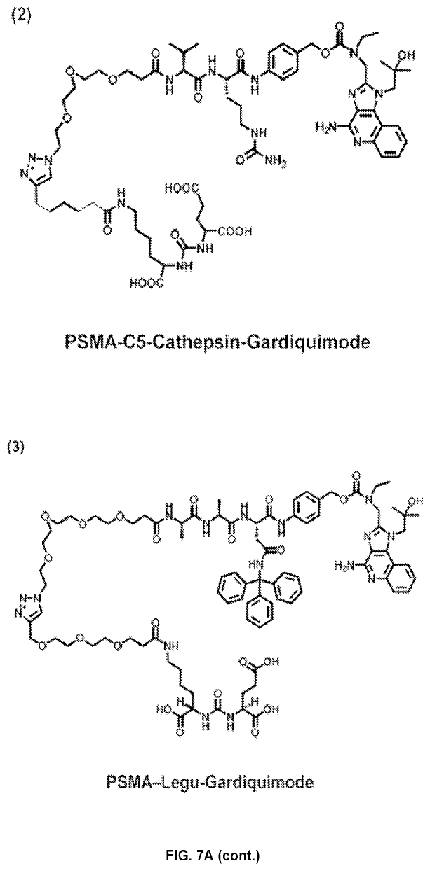

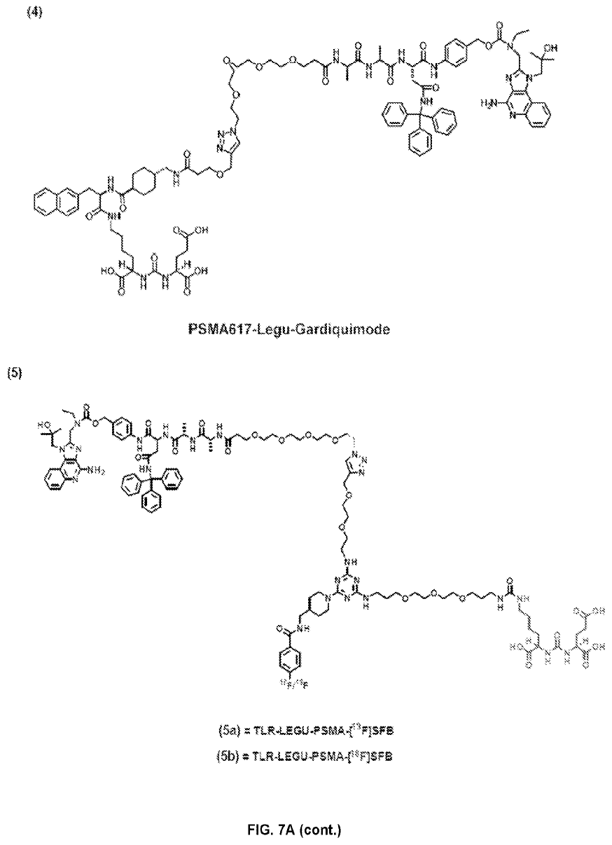

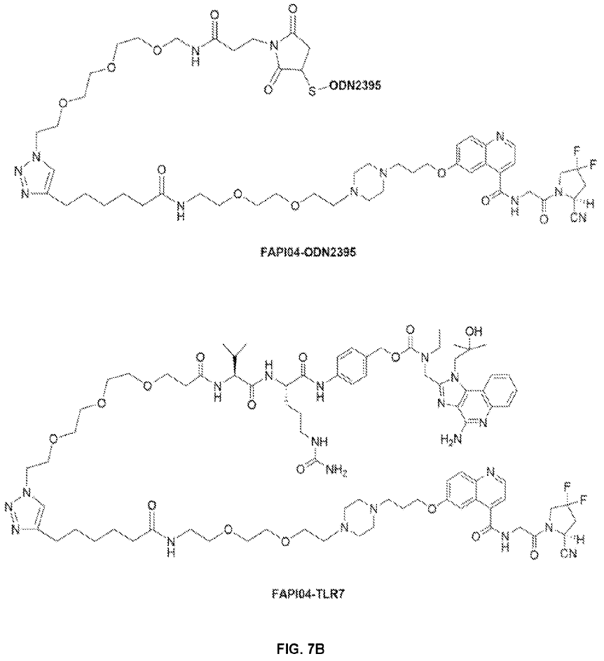

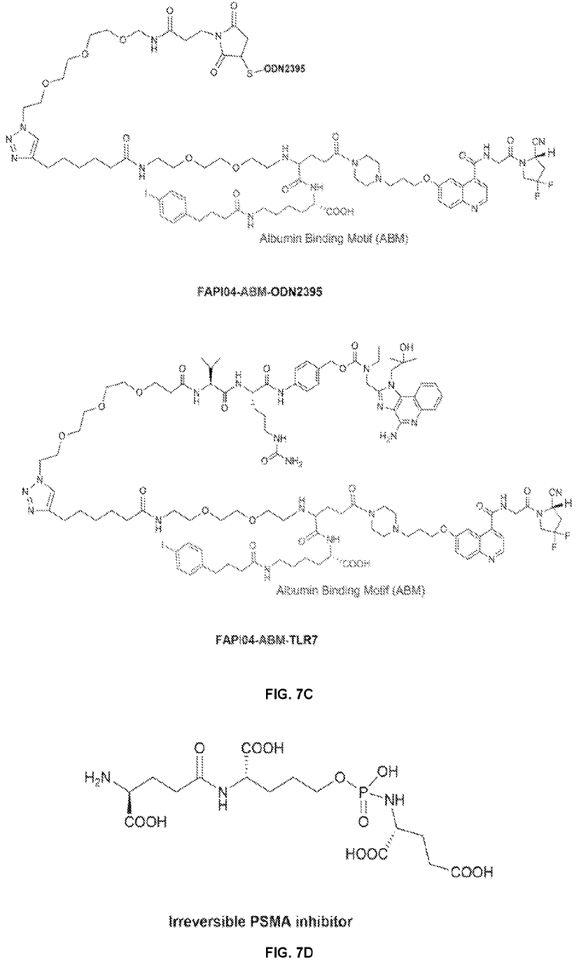

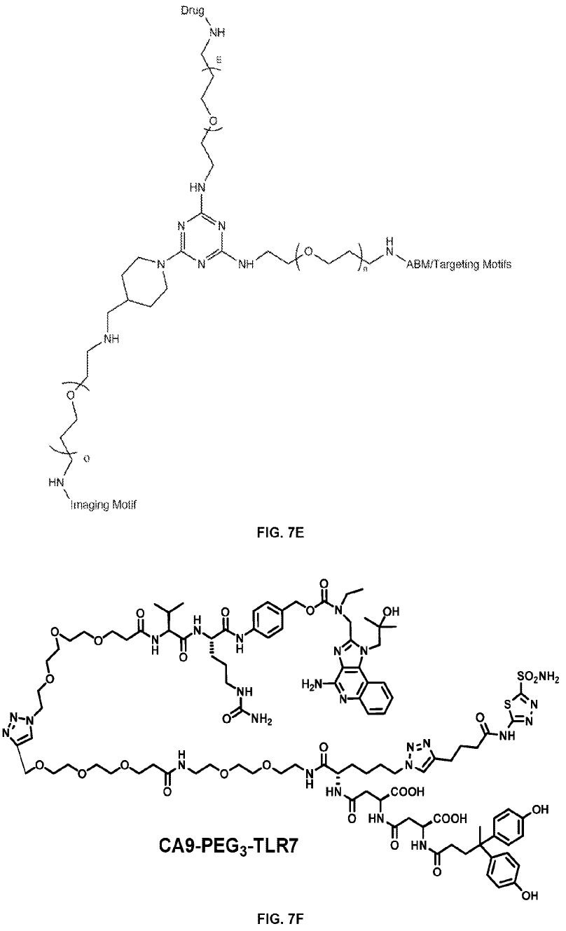

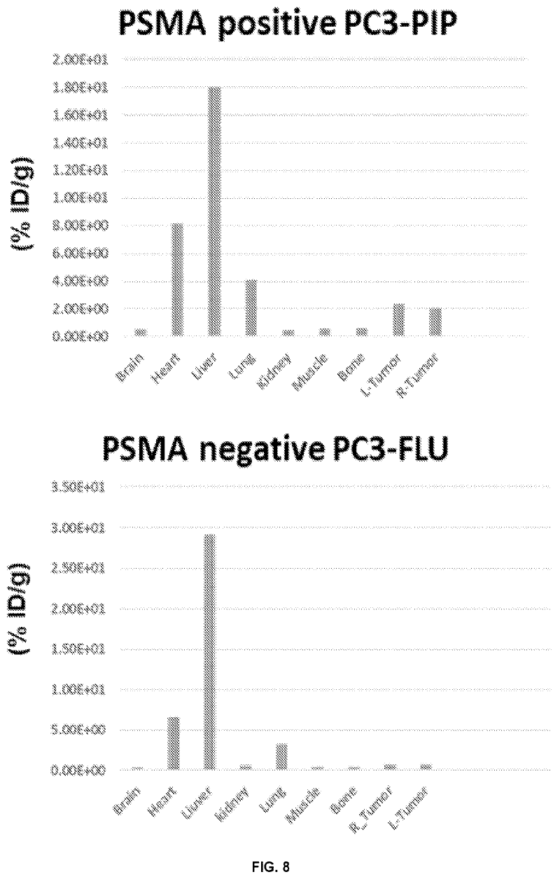

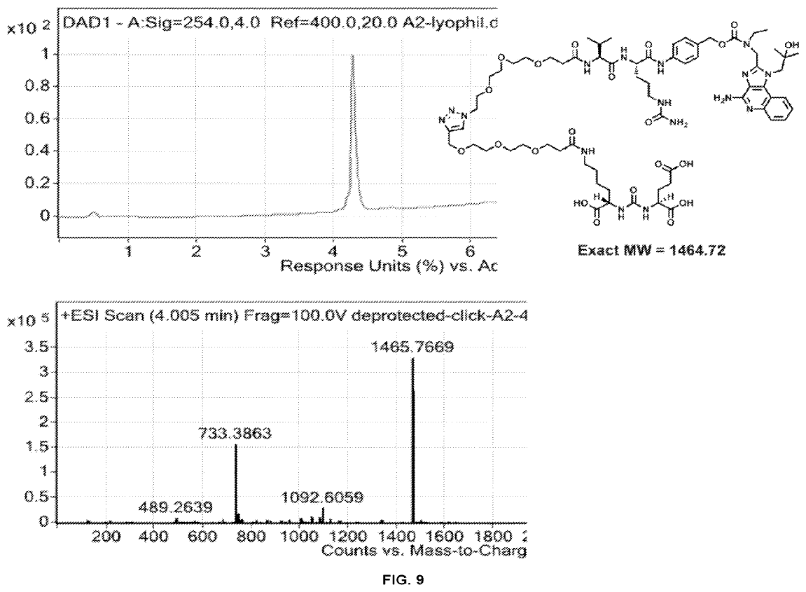

Exemplary prodrugs comprise, e.g., (i) one or more targeting moieties that selectively binds to an extracellular antigen such as an extracellular tumor antigen, (ii) one or more an enzyme-cleavable linkers, and (iii) one or more innate immune system activators. Examples of prodrugs are shown in FIG. 7.

Radiolabel moieties can be used for the labeling of the prodrug with a radionuclide. The labeling can be useful for imaging, for radiation therapy, or both for imaging and radiation therapy. A radiolabel moiety can be linked to a prodrug by a prosthetic group.

A prosthetic group can include bifunctional chelators such as DOTA for radiolabeling with a select radionuclide for imaging by PET or SPECT.

In some embodiments, a prosthetic group can comprise a moiety for labeling with a radionuclide selected from ¹⁸F, ¹²³I, ¹²⁴I, ¹³¹I, ⁷⁵Br or ⁷⁶Br.

In other embodiments, a prosthetic group can comprise a bifunctional chelator for radiolabeling with a radionuclide for imaging selected from ⁶⁷Ga, ⁶⁸Ga, ⁶⁰Cu, ⁶¹Cu, ⁶²Cu, ⁶⁴Cu, ⁸⁹Zr, ¹⁷⁷Lu, ¹¹¹In, and ^{99m}Tc.

In some embodiments, a prosthetic group can comprise a bifunctional chelator for radiolabeling with a radionuclide for radiation therapy selected from ⁶⁷Cu, ¹⁷⁷Lu, ⁹⁰Y, ²²⁵Ac, and ²²⁷Th.

A prodrug can comprise a radiolabel so that the prodrug can be used for imaging, radiation therapy, or both for imaging and radiation therapy. A prosthetic group can be linked to a prodrug by a second linker.

For example, the prosthetic group can be used for functional imaging. In some embodiments, the functional imaging can be positron emission tomography (PET), single-photon emission computed tomography (SPECT).

A second innate immune system activator can comprise a proinflammatory molecule. Any proinflammatory molecule can be used, including TLR agonists and STING agonists, for example. A second activator (or third, fourth, fifth or more) of the innate immune system can be the same as or different from a first activator of the innate immune system. For example, a first activator of the innate immune system can be a TLR agonist and a second innate immune system activator can be a different TLR agonist. As another example, a first activator of the innate immune system can be a TLR agonist and a second innate immune system activator can be the same TLR agonist. As yet another example, a first activator of the innate immune system can be a STING agonist and a second innate immune system activator can be a different STING agonist. As a further example, a first innate immune system activator can be a STING agonist and the second innate immune system activator can be the same STING agonist. As yet a further example, a first innate immune system activator can be a STING agonist and a second innate immune system activator can be a TLR agonist. As yet another example, a first innate immune system activator can be a TLR agonist and a second innate immune system activator can be a STING agonist.

The second (or third, fourth, fifth or more) innate immune system activator can be linked to a prodrug provided herein by another linker as described herein. A linker for linking the chelating contrast agent and/or the radionuclide containing moiety and the linker for linking the innate immune system activator(s) to the prodrug can be the same or different from each other. These linkers can also be the same or different from the linker for linking a first innate immune system activator to a prodrug. An example, an immunogenic molecule such as an innate immune system activator can be linked to the prodrug by an enzyme-cleavable linker and the targeting moiety such as a PSMA, CA9, or aVb3 molecule can be linked by a diamine linker. An exemplary prodrug can comprise i) a targeting moiety that specifically binds to an extracellular antigen such as an extracellular tumor antigen; (ii) an enzyme-cleavable linker; (iii) an innate immune system activator and (iv) a radionuclide containing moiety. An example of (5a) TLR-LEGU-PSMA-[¹⁹F]SFB(5b) TLR-LEGU-PSMA-[¹⁸F]SFB is shown in FIG. 7A.

Methods of Treating Cancer

Methods of treating cancer in a subject are provided. Methods can comprise administering an amount of a prodrug provided herein effective for treating the cancer to a subject. Any of the prodrugs provided herein can be used for treating cancer.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a disease or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes inhibiting the disease, i.e., arresting its development, and relieving the disease, i.e., causing regression of the disease. In an embodiment, treatment causes the reduction or alleviation of one or more symptoms of a disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. In an embodiment, administration of the compounds herein can prevent a disease from occurring in a subject which can be predisposed to the disease or at risk of acquiring the disease but has not yet been diagnosed as having it. In some cases, for prophylactic benefit, prodrugs or compositions are administered to a subject at risk of developing a particular disease, or to a subject reporting one or more of the physiological symptoms of a disease, even though a diagnosis of the disease may not have been made. The methods of the present disclosure can be used with any mammal or other animal. In some cases, the treatment can result in a decrease or cessation of one or more symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “subject” refers to any individual or patient on which the methods disclosed herein are performed. The term “subject” can be used interchangeably with the term “individual” or “patient.” The subject can be a human, although the subject can be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “effective amount” or “therapeutically effective amount” refers to that amount of a prodrug or composition described herein that is sufficient to affect the intended application, including but not limited to disease treatment, as defined herein. The therapeutically effective amount can vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular prodrug or composition chosen, the dosing regimen to be followed, whether it is administered in combination with other compounds, timing of administration, the tissue to which it is administered, and the physical delivery system in which it is carried.

Any cancer can be treated using the methods provided herein, including kidney cancer, renal cancer, prostate cancer, lung cancer, colon cancer, rectal cancer, urinary bladder cancer, melanoma, oral cavity cancer, pharynx cancer, pancreatic cancer, uterine cancer, thyroid cancer, skin cancer, head and neck cancer, breast cancer, cervical cancer, ovarian cancer, brain cancer, or hematopoietic cancer, for example. In some embodiments, treating cancer further comprises administering a prodrug prior to, simultaneously with, or following administration of immunotherapy. For example, a prodrug can be administered about 1, 30, 60, 90 minutes or more after or before administration of immunotherapy. In another example, a prodrug can be administered about 2, 5, 12, 24, 36, 48 hours or more after or before administration of immunotherapy. In another example, a prodrug can be administered about 3, 5, 7, 14, 21, 27 days or more after or before administration of immunotherapy. Any immunotherapy can be administered for the treatment of cancer. Immunotherapy includes, for example, treatment with activation immunotherapies and treatment with suppression immunotherapies. Activation immunotherapies elicit or activate an immune response, while suppression immunotherapies reduce or suppress an immune response. Immunotherapy can include treatment with immune modulators, such as interleukins, cytokines, chemokines, immunomodulatory imide drugs (IMiDs), and others. Any interleukin, cytokine, chemokine, or immunomodulatory imide drug (IMiD) can be used for immunotherapy. Exemplary interleukins for immunotherapy include IL-1, IL-2, IL-3, IL-4, IL-5, IL-6, IL-7, IL-8, IL-10, IL-12, IL-15, IL-18, IL-21, and IL-23. Exemplary cytokines for immunotherapy include interferons TNF-α, TGF-β, G-CSF, and GM-CSF. Exemplary chemokines for immunotherapy include CCL3, CCL26, and CXCL7. Exemplary IMiDs include thalidomide and its analogues lenalidomide, pomalidomide, and apremilast. Other immunomodulators include cytosine phosphate-guanosine, oligodeoxynucleotides, and glucans, for example.

Cancer immunotherapy generally involves stimulation of the immune system to destroy cancer cells and tumors. Exemplary cancer immunotherapy includes CAR T-cell therapy that introduces chimeric antigen receptors (CARs) to a patient's T cells to generate CAR-T cells. CAR-T cells are then introduced into the patient's bloodstream to treat cancer by adoptive cell transfer (ACT). CARs generally include antigen recognition domains that can target antigens expressed on the cell surface of cancer cells and one or more signaling domains. Thus, CAR-T cells can target and destroy cancer cells that express a target antigen. Exemplary CAR-T cell therapies include tisagenlecleucel (KYMRIAH) and axicabtagene ciloleucel (YESCARTA).

Cancer immunotherapy can also comprise TCR therapy, another type of ACT. Similar to CAR-T cell therapy, T cells are taken from a patient, reengineered, and introduced to the patient. A further type of ACT includes tumor-infiltrating lymphocyte (TIL) therapy. TILs from a patient are isolated from a patient's tumor tissue and expanded in vitro, followed by introduction into the patient.

Yet another type of cancer immunotherapy includes treatment with monoclonal antibodies. Monoclonal antibodies for use in immunotherapy can be naked, i.e., non-conjugated, or conjugated, i.e., have a chemotherapy drug or radioactive particle attached to them. In addition to monoclonal antibodies, other molecules such as interleukins and cytokines, for example, can be conjugated for targeting cancer cells. As an example, denileukin diftitix (ONTAK) includes IL-2 attached to diphtheria toxin. Furthermore, monoclonal antibodies for cancer immunotherapy can be bispecific, i.e., designed to recognize and bind to two different proteins. Thus, bispecific monoclonal antibodies can recognize more than one antigen on the surface of a cancer cell, for example. As another example, a bispecific antibody can recognize a protein or antigen on a cancer cell and a protein or antigen on an immune cell, thereby promoting the immune cell to attack the cancer cell.

Exemplary monoclonal antibodies for treating cancer include alemtuzumab (CAMPATH®), trastuzumab (HERCEPTIN®), ibritumomab tiuxetan (ZEVALIN®), brentuximab vedotin (ADCETRIS®), ado-trastuzumab emtansine (KADCYLA®), blinatumomab (BLINCYTO®), bevacizumab (AVASTIN®), and cetuximab (ERBITUX®).

Further cancer immunotherapies include cancer vaccines that elicit an immune response against cancer cells.

Yet another cancer immunotherapy comprises checkpoint inhibitor therapy. Checkpoint inhibitor therapy is a form of cancer treatment that uses or targets immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. Without being limited by theory, PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, for example, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

PD-L1, an immunosuppressive PD-1 ligand, is expressed in several cancers and in many tumor cells. Inhibition of the interaction between PD-1 and PD-L1 can enhance T-cell responses in vitro and mediate preclinical antitumor activity. Monoclonal antibodies targeting PD-1 can boost the immune system for the treatment of cancer.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but generally upregulated in conventional T cells after activation, especially in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. Without being limited by theory, CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

Several checkpoint inhibitors can be used to treat cancer. PD-1 inhibitors include Pembrolizumab (KEYTRUDA®) and Nivolumab (OPDIVO®), for example. PD-L1 inhibitors include Atezolizumab (TECENTRIQ®), Avelumab (BAVENCIO®) and Durvalumab (IMFINZI®), for example. CTLA-4 inhibitors include Iplimumab (YERVOY®), for example. Other checkpoint inhibitors include, for example, an anti B7-H3 antibody (MGA271/Enoblituzumab), an anti-KIR antibody (Lirilumab) and an anti-LAG3 antibody (BMS-986016). Any checkpoint inhibitor can be used in the methods described herein.

Prodrugs provided herein can be administered with any immunotherapy or with any combination of immunotherapies in the methods provided herein. Prodrugs can be synergistic with any immunotherapy or combination of immunotherapies that it is co-administered with. As used herein, “synergism” means that the total effect of two or more drugs is greater than the sum of the individual effects of each drug.

In some embodiments, the immunotherapy comprises an interleukin, a cytokine, a chemokine, an immunomodulatory imide drug, CAR-T cells, TCR therapy, a monoclonal antibody, a cancer vaccine, a checkpoint inhibitor, or combinations thereof.

Types and Doses of Administration

Prodrugs can be administered by any route, including orally, intraduodenally, parenterally (including intravenous, subcutaneous, intramuscular, intravascular or by infusion), topically or rectally. Prodrugs can be administered at any dose and with any frequency to produce a desired treatment outcome. For example, a prodrug can be administered in the range of about 0.001 to about 1000 mg/kg body weight/day. A prodrug can also be administered at a dose of about 0.1, about 0.5, about 1 mg, about 5 mg, about 10 mg, about 20 mg, about 30 mg, about 40 mg, about 50 mg, about 60 mg, about mg, about 80 mg, about 90 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, and any number or range in between. Administration can be once per day, two times per day, three times per day, four times per day or more. Prodrugs provided herein can be administered for 1 week, 2 weeks, 3 weeks, 4 weeks, 5 weeks, 6 weeks, 7 weeks, 8 weeks, 9 weeks, 10 weeks, 11 weeks, 12 weeks, or more. Administration for a particular period of time at a particular dose given one or more times a day can constitute a cycle of treatment. A cycle of treatment can be repeated after a recovery time of 1 week, 2 weeks, 3 weeks, 4 weeks, weeks, 6 weeks, 7 weeks, 8 weeks, 9 weeks, 10 weeks, 11 weeks, 12, weeks, or more. A single cycle or more than one cycle of treatment with a prodrug can be administered. Any number of cycles can be administered for a course of treatment. A single course or more than one course of treatment can be administered.

Methods of Imaging

A method can comprise, for example administering to a subject a prodrug comprising (i) one or more (1, 2, 3, 4, 5, or more) targeting moieties that specifically bind to an extracellular antigen such as an extracellular tumor antigen; (ii) one or more (e.g., 1, 2, 3, 4, 5, or more) enzyme-cleavable linkers; (iii) one or more (e.g., 1, 2, 3, 4, 5, or more) immune system activators. The enzyme-cleavable linker can covalently link a targeting moiety to an innate immune system activator. A prodrug can further comprise a chelating moiety. Immunotherapy can optionally be administered after, simultaneously with, or before administration of the prodrug. For example, a prodrug can be administered about 1, 30, 60, 90 minutes or more after or before administration of immunotherapy. In another example, a prodrug can be administered about 2, 5, 12, 24, 36, 48 hours or more after or before administration of immunotherapy. In another example, a prodrug can be administered about 3, 5, 7, 14, 21, 27 days or more after or before administration of immunotherapy. Imaging can include performing positron emission tomography (PET), computed tomography (CT), or a combination thereof on the subject.

Any immunotherapy can be administered after, simultaneously with, or before administration of the prodrug. In some embodiments, immunotherapy comprises administration of an interleukin, a cytokine, a chemokine, an immunomodulatory imide drug, CAR-T cells, TCR therapy, a monoclonal antibody, a cancer vaccine, a checkpoint inhibitor, or combinations thereof.

Methods of Monitoring Treatment of Cancer

Treatment of cancer can be monitored using methods provided herein. A method can comprise, for example, administering to the subject a prodrug comprising (i) one or more targeting moieties (e.g., 1, 2, 3, 4, 5, or more) that specifically bind to an extracellular antigen such as an extracellular tumor antigen; (ii) one or more (e.g., 1, 2, 3, 4, 5, or more) enzyme-cleavable linkers; and (iii) one or more innate immune system activators (e.g., 1, 2, 3, 4, 5, or more). An enzyme-cleavable linker can covalently link the targeting moiety to the innate immune system activator. A prodrug can further comprise a chelating contrast agent, which modifies the distribution of gadolinium within the body to overcome its toxicity while maintaining its contrast properties for imaging purposes. Immunotherapy can optionally be administered to the subject after, simultaneously with, or before administration of the prodrug. For example, a prodrug can be administered about 1, 30, 60, 90 minutes or more after or before administration of immunotherapy. In another example, a prodrug can be administered about 2, 5, 12, 24, 36, 48 hours or more after or before administration of immunotherapy. In another example, a prodrug can be administered about 3, 5, 7, 14, 21, 27 days or more after or before administration of immunotherapy. Imaging can include performing positron emission tomography (PET), computed tomography (CT), or a combination thereof on the subject.

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook, et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, N Y (1992); Guthrie and Fink, Guide to Yeast Genetics and Molecular Biology, Academic Press, N Y (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### Example 1

This example describes the design of TLR agonist prodrugs.

Because systemic delivery of TLR agonists is undesirable due to dose limiting toxicity, general inflammatory response of the host, especially for TLR7 agonists, and/or limited efficacy, delivery of TLR agonists is generally limited to local or intra-tumoral injections that have to be repeated on a weekly basis, for example. Clinical development of TLR agonists is further hampered by additional delivery barriers such as, for example, risk of bleeding, organ damage, infection and cost, in particular with deep seated metastases like the lung, which is the most common site of recurrence in kidney cancer, for example. Tumor heterogeneity presents another reason for the limited value of local or intra-tumoral delivery of TLR agonists. For example, metastatic kidney cancer like most cancers, has undergone clonal evolution. Some tumor deposits will be immunologically different than others. Accordingly, limiting injection to one to two tumors per patient will generally not result in a maximum potential benefit compared to causing inflammation in all metastases in all patients while preventing a general inflammatory state outside of the tumor environment. The design of proinflammatory prodrugs for the delivery of, for example, TLR agonists allows for systemic injection of the prodrug and prodrug activation in target cells, thus overcoming the undesirable effects of systemic TLR agonist delivery.

TLR Agonists

Proinflammatory prodrugs can be designed for the delivery of, for example, TLR agonists. In an embodiment, agonists of any TLR can be used in the design of proinflammatory prodrugs. Expression of TLR7 and TLR9 and exemplary agonists of TLR7 and TLR9 are shown in Table 2.

TABLE 2
Expression and Exemplary Agonists of TLR7 and TLR9

| | | |
|---|---|---|
| TLR7 | Expression in cells of the | TLR 7 agonists are small |
| | immune system (macrophages, | diffusible adenine Examples: |
| | neutrophils, cells). | Imiquimod, Resiquimod, |
| | | Gardiquimod |
| TLR9 | Expression in cells of the | TLR9 agonists are small |
| | immune system (macrophages, | oligonucleotides with |
| | neutrophils, cells). Expressed | phosphorothioate backbones. |
| | also in kidney cancer cells and | Examples: ODN-2395, |
| | tumor endothelial cells. | CMP-001, MGN1703 |

TLR7 and TLR9 agonist prodrugs can be delivered to cancer metastases, such as clear cell kidney cancer metastases, through an infusion. All or nearly all cancer metastases can be reached by the prodrug, resulting in significantly improved immunotherapy.

Targets for Prodrug Delivery

The targeted delivery of prodrugs in kidney cancer can include the use of validated surface proteins that are a) specific for kidney cancer cells or the tumor's microenvironment, b) expressed in the majority of clear cell kidney cancer cases and/or c) have validated probes suitable for drug development. Clear cell kidney cancer, which represents the vast majority of metastatic kidney disease, has distinct high quality markers such as carbonic anhydrase 9 (CA9) and Prostate-Specific Membrane Antigen (PMSA), which can be used for selective and effective delivery of prodrugs.

Carbonic anhydrase 9 (CA9) is a cell surface protein which is highly and specifically expressed on clear cell renal cell carcinoma cells. Clear cell kidney cancer is molecularly defined by loss, mutation or silencing of the VHL protein, which leads to the dramatic upregulation of hypoxia induced transcription factors (HIF1/2) which in turn leads to the very high expression levels of CA9 in 95% of clear cell kidney cancers. Thus, CA9 is a high value marker/target with only limited expression in normal tissues. For example, CA9 expression is minimal in the intestine and low in the bililary tree.

Prostate-Specific Membrane Antigen (PSMA) is expressed on the surface of prostate cancer and on the neo-vasculature of many solid tumors (e.g., kidney, breast and lung cancer). PSMA can be used for imaging of metastatic prostate cancer and its treatment with radioactive probes, demonstrating the clinical utility and value of small molecular probes which can deliver radiation to tumors. The rich and dense neovasculature of clear cell kidney cancer reliably expresses high levels of PSMA.

Without being limited by theory, in contrast to PSMA-targeted delivery of radioactively labelled probes, TLR engagement is reversible and can be counteracted by withholding the drug or by administering corticosteroids. In addition, at least for TLR7 agonism, the primary targets are innate immune cells that unlike T cells are present in large numbers in most cancers, but not abundantly present in normal, physiological tissues, thus reducing the risk of adverse events.

CA9 and PSMA are expressed in clear cell kidney cancer and that the bulk of cancer cells can be targeted via CA9 while the cancer-associated vasculature can be targeted via PSMA.

Linkers

Inadequate linker design has led to the failure of numerous prodrug approaches. Therefore, the stability of a prodrug in serum and the timely and appropriate release inside the target cells should be considered for prodrug design. Additionally, TLR7 agonists are small molecules that require the traceless release of their linker to regain their pharmacological activity. A peptide linker valine-citrulline (VAL-CIT), a bona-fide cathepsin B cleavable substrate, was chosen for testing. Since cathepsin B is only present in lysosomes, the internalized prodrug is cleaved only inside the cell, which in turn destabilizes a self-immolating para-amino-benzyl alcohol, ultimately releasing a TLR7 agonist such as gardiquimod in a traceless fashion. This design takes into account the desire to develop prodrugs with highest translational potential to maximize patient benefits.

There is less need for a sophisticated linker system for TLR9 agonists/oligonucleotides. These molecules are fairly large in size and can be covalently linked without losing stimulatory activity. In an embodiment, a prodrug can be released from its surface receptor in the lysosome, either because of acidic conditions or because of the eventual proteolytic degradation of the CA9 or PSMA surface receptor. In turn, the oligonucleotide can bind to its cognate receptor, i.e. TLR9, and engage the TLR signaling pathway.

### Example 2

This example describes design, synthesis, and testing of a proinflammatory prod rug PSMA-TLR7.

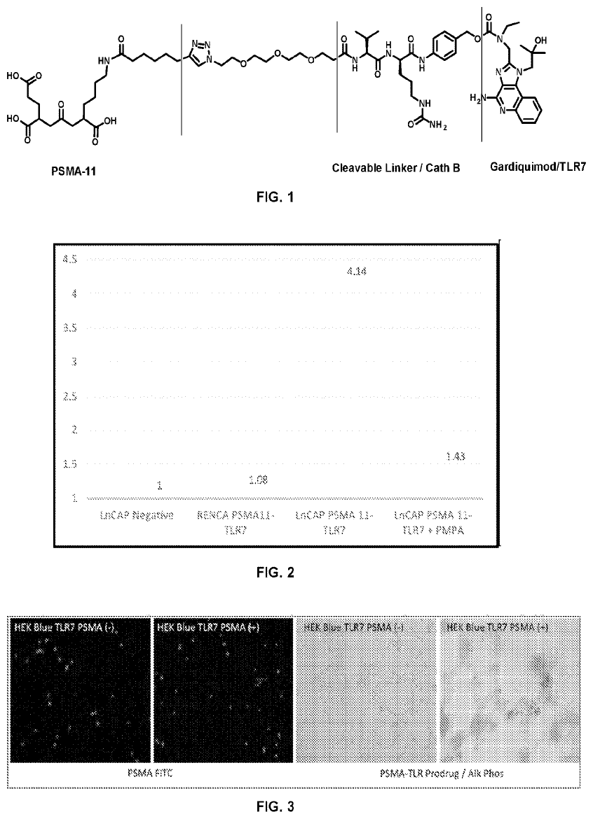

The prodrug PSMA-TLR7 (FIG. 1) was synthesized, purified, and tested in cell culture with cell lines that either express PSMA (LnCAP, HEK293 Blue TLR7 PSMA) or don't express PSMA (RENCA, HEK Blue TLR7). RENCA and LnCAP cells were exposed to control, the prodrug or prodrug plus a potent PSMA inhibitor, i.e., 2-phosphonomethylpentanedioic acid (PMPA), which competes with the PSMA probes for their binding site. After 24-48 hours cells were harvested and extracted with ethanol. Extracts were dried and then resuspended into cell culture media which was then applied to a PSMA negative TLR7 reporter cell line for at least 24 hours. Colorimetric substrate for alkaline phosphatase activity was applied and measured using spectrophotometry. The data show that PMPA was able to compete with the prodrug, suggesting specific uptake via cell surface PSMA (FIG. 2).

The PSMA negative TLR7 HEK Blue cell line was stably transduced with a lentivirus carrying the human PSMA gene. Transduction led to subset of around 5% of PSMA positive cells (FIG. 3, PSMA FITC expression). Both PSMA negative and PSMA positive cells were exposed to the PSMA11-TLR7 prodrug together with the colorimetric alkaline phosphatase substrate. No cells in the PSMA negative cell line reported TLR7 target engagement. However, in the mixed population, cells reporting TLR7 receptor stimulation were clearly present.

Together, the data suggest not only target/PSMA specific uptake of the prodrug, but also indicate significant prodrug stability in serum containing media. Without being limited by theory, serum stability is the likely result of the cathepsin B selective prodrug response. The azide-pegylated-Val-Cit-PAB-PNP linker is very stable in serum and cleaved only intracellularly by cathepsin B, allowing for traceless release of small molecules such as gardiquimod. Furthermore, these results show the successful design and testing of proinflammatory prodrugs. Without being limited by theory, the prodrugs are designed to bind to two important surface markers for clear cell kidney cancer or its tumor microenvironment, lead to their selective release in the lysosomes of the target cells and then engage the TLR pathway leading to a significant inflammatory response (FIG. 4).

### Example 3

This example describes design of TLR7 and TLR9 Prodrugs.

TLR7 and TLR9 prodrugs that target PSMA and CA9 expressing cells were designed. Without being limited by theory, prodrugs that target PSMA are designed to target the kidney vasculature, while prodrugs that target the CA9 cell surface protein of clear cell kidney cancer are designed to target tumor cells.

The chemistry of the targeting components (i.e., PSMA and CA9) as well as the inflammatory payloads were designed in a modular fashion. This allows for simple “click synthesis” to generate four different molecules: PSMA-TLR7, PSMA-TLR9, CA9-TLR7, and CA9-TLR9. Click-synthesis describes the efficient and selective copper-mediated coupling reaction between an alkyne and an azide group. Both the PSMA targeting glutamate-urea-lysine backbone and the dual motif CA9 ligand are reacted with oct-7-ynoic acid using a standard hexafluorophosphate benzotriazole tetramethyl uronium (HBTU) mediated peptide bond formation. This equips both targeting moieties with a hydrophobic linker, which is not only important for PSMA binding but also introduces the required alkyne group for subsequent click-synthesis.

The linker system varies with the respective inflammatory payload. Gardiquimod is a potent small molecule TLR7 agonist which has no activity once it is covalently linked to a larger molecule. This molecule can be delivered with a reversible/cleavable linker system. While there are a number of possible options to choose from (e.g. disulfide bonds), a very stable, self-immolating system that allows for the traceless release of gardiquimod was chosen for testing. An azide-pegylated-Val-Cit-PAB-PNP linker system (Broadpharm) was used. This linker is extremely stable in human serum and is only cleaved by cathepsin B inside lysosomes, which is where the TLR7/9 receptors are located. Importantly, gardiquimod has a secondary aliphatic amine that readily reacts with the nitrophenylcarbonate group of the linker with high yield.

TLR9 agonist ODN-2395 was chosen for testing. ODN-2395 is a type III CpG oligonucleotide with a stabilizing phosphorothioate backbone. This oligonucleotide has potent TLR9 stimulating activity in mouse and human cells. In contrast to gardiquimod, TLR9 oligonucleotides can be covalently linked to other molecules without losing activity. Therefore, a simpler linker system was chosen for the TLR9 agonist. ODN 2395 (5′-TCGTCGTTTTCGGCGCGCGCCG-3′) (SEQ ID NO:1) is custom manufactured with a 5′ thiol modification (IDT DNA) and then linked in a simple Michael addition to a commercially available azide-peg-maleimide linker (Kerafast). This payload can then be clicked to either the PSMA or the CA9 targeting moiety as above. All synthetic products are purified using standard chromatographic procedures and their identity confirmed with mass spectrometry.

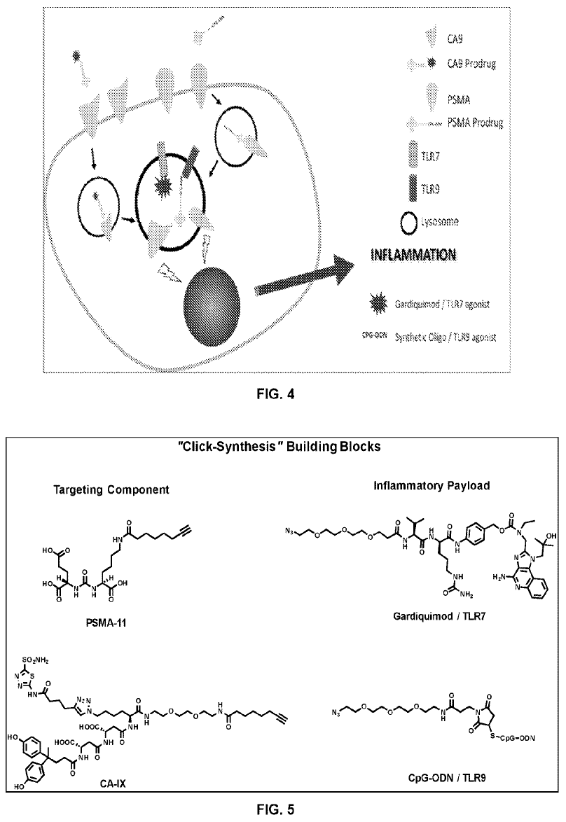

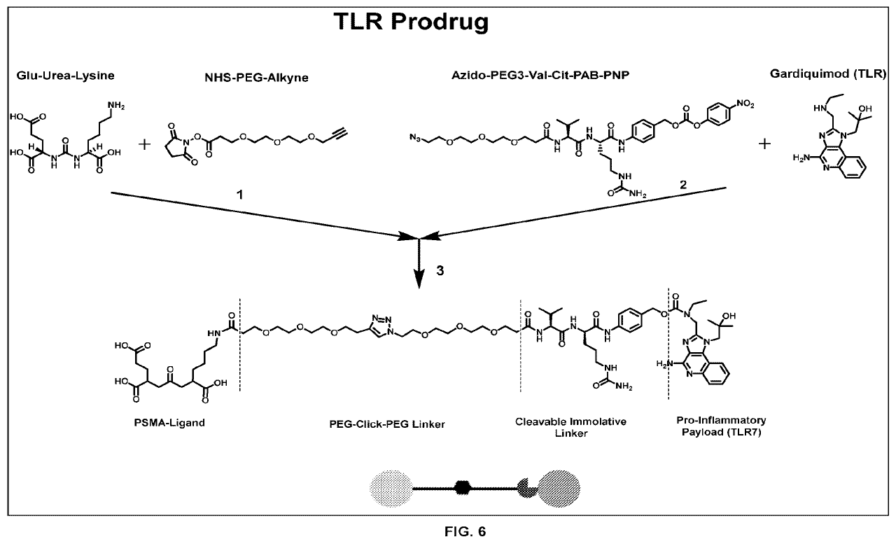

FIG. 5 shows the modular design of prodrug building blocks. Alkyne groups on the targeting components are linked to the azides of the TLR payloads with copper-(I)-mediated “click-synthesis.” A strategy for synthesis of a TLR7 prodrug targeting PMSA is shown in FIG. 6 at top. Four prodrugs targeting PSMA and CA9 with TLR7 and TLR9 agonists are shown in FIG. 6 at bottom. Structures of PSMA targeting TLR7 prodrugs are shown in FIG. 7 (Prodrugs (1)-(4)).

### Example 4

This example describes in vitro characterization of prodrugs.

The stability of the prodrugs in serum, their binding affinity to their target, their effect on TLR reporter activity and a murine kidney cancer cell line can be determined. Without being limited by theory, immunotherapy prodrugs that target the cell surface proteins CA9 and PSMA can enter cells, release their cargo, and stimulate TLR pathways in target cells.

Serum stability assay. Prodrugs can be incubated at a concentration of 10 microM in PBS, cell culture media with 10% FBS, as well as 100% human and 100% mouse serum at 37° C. for up to 1 week. Daily samples are removed, extracted with acetonitrile and submitted for mass spectrometry. Samples are submitted to LC-MS and compared to standard curves of the free TLR7/unconjugated TLR9 agonist and their intact prodrugs. The intra- and inter-day run precision and accuracy of replicate assays are assessed by percentage of the coefficient of variations (Graph Pad).

Binding affinity of the Prodrugs. A competitive fluorescence polarization assay can be used to determine the binding affinity to the respective targets. The targeting modules of the PSMA and CA9 prodrug are clicked to an azide FITC derivative and purified. Recombinant CA9 and PSMA proteins (RnD Systems) are incubated with serially diluted concentrations of the purified prodrugs. FITC labeled ligands are added and fluorescence polarization measurements are performed on an EnVision multi-modal microplate reader (PerkinElmer, Inc., UT Southwestern HTS core). Experiments are carried out in triplicate and the concentration resulting in 50% response (IC50) is calculated in GraphPad Prism (GraphPad Software, La Jolla, CA) using the sigmoidal dose-response regression function. This information is useful for future optimization considerations of the prodrugs.

Target Engagement of the TLR7/TLR9 prodrugs. Gardiquimod and ODN-2395 both engage the human and mouse TLR7 and TLR9 receptor, respectively. Human TLR7/TLR9 HEK293 Blue reporter cell lines (Invivogen) can be transduced with lentivirus (Clontech, Addgene), forcing the stable expression of cell surface PSMA or CA9. Cells can be sorted by flow cytometry and then exposed to prodrugs at various concentrations for 24-48 hours. Colorimetric detection of induced alkaline phosphatase activity is performed. EC50 concentrations are determined using Graphpad as above.

Effects of prodrugs on a murine kidney cancer cell line. Since the RENCA cell line, which will be used in the animal studies, has been reported to express at least TLR7, the direct effect of the prodrugs on wildtype as well as PSMA and CA9 transduced cells can be determined. Readouts comprise effects on cell proliferation (cell numbers) as well as induction of cytokines like tumor necrosis factor alpha (TNF-a) and interferon-alpha (IFN-a) in the supernatant determined by ELISA (RnDSystems).

Preliminary data for PSMA-TLR7 (Example 2) has demonstrated the feasibility of the synthetic approach outlined above. Additionally, data suggests the specific binding and uptake of the PSMA prodrug as well as the release of free gardiquimod. The same release linker as for the gardiquimod approach can be used if the covalently linked TLR9 does not exhibit adequate biological activity.

Sufficient prodrug material is generated for animal studies. At least 10-20 mg of each prodrug is prepared for in vivo studies.

### Example 5

This example describes determination of the efficacy and immunological effects of TLR7 and TLR9 prodrugs in a syngeneic mouse model of renal cell carcinoma.

The only existing, transplantable syngeneic renal cell carcinoma model in mice (Balb/C) is the RENCA model. While this tumor model is not of classic clear cell histology, it has two salient features, which make it the best preclinical model available: 1) It has been validated in several preclinical immunotherapy studies, primarily with IL-2 and 2) it is resistant to PD1 monotherapy, which is a problem the prodrug approach is intended to overcome. Additionally, the mouse tumor vasculature does not express PSMA. Therefore, PSMA expression on the mouse tumor vasculature is emulated with a PSMA positive cell line.

PSMA and CA9 transduced cell lines can be used. Tumors are placed subcutaneously and allowed to grow to 200-300 mm 3 within 7-10 days. Typically, groups of 10 mice are inoculated, allowing for some attrition. 8 cohorts (2 per prodrug) are then exposed to two different dose levels of prodrugs, e.g., 1 mg/kg and 10 mg/kg, which is injected IV twice weekly. One untreated cohort and one cohort treated with free drug will serve as controls. Animals are monitored for toxicity and weight loss and blood is collected for plasma cytokines. Subsequent experiments combine the four different prodrug cohorts at their highest tolerated dose with an anti-mouse PD1 inhibitor (BioXcell) given IP and a cohort which combines a TLR7 with a TLR9 prodrug in a PSMA/CA9 mixed tumor. For drug distribution studies four cohorts of four animals are exposed to each prodrug and prodrug distribution levels are determined six hours after injection of the tumor, liver and kidney using LC-MS as above. Primary endpoint will be tumor response defined as regression. Tumor volume is assessed using caliper measurements (tumor volume=½ (length×width²)) and determined three times per week. Ten mice/group are generally adequate to determine a RR of 30% in combination treated mice versus monotherapy treated mice with an alpha of 0.05 and a beta of 0.1. Additionally, tumor-infiltrating immune cells can be characterized using optimized multicolor phenotyping panels for mouse immune cells (ThermoFisher). The cells are analyzed in a Flow Cytometry Core facility using a FACSCalibur flow cytometer (BD Biosciences). Data are analyzed with FlowJo software. For comparison of cell numbers, including CD8 cells, CD4 cells, NK cells, and others, and percentages between treatment groups, an unpaired t test or ANOVA as appropriate is used. P values <0.05 are considered significant.

Additional cells that can be used include the mouse prostate cancer cell line MYC-CAP, for example. MYC-CAP cells are transduced with lentiviral vectors for expression of PSMA. Expression of TLR7 in MYC-CAP cells is determined by qPCR. Without being limited by theory, the TLR7 agonist is released in cancer cells and then diffuses freely into myeloid bystander cells such as myeloid-derived suppressor cells and dendritic cells, creating a proinflammatory field effect in an animal model, for example. Direct in vitro effects of the prodrug on PSMA-positive and PSMA-negative MYC-CAP cells with respect to proliferation and the induction of cytokines such as type I interferon, for example, is determined by MTT assay and ELISA, respectively. PSMA-positive and PSMA-negative MYC-CAP cells are transplanted to male FVB mice by subcutaneous injection. Tumors will be allowed to grow to 200-300 mm³ and the effect of treatment with prodrugs will be analyzed as described above.

These studies are designed to show that the systemic treatment with TLR prodrugs, in particular together with PD1 inhibitors, will lead to T cell infiltration and antitumor effects. The combination of two different TLR agonists can be superior in this setting. If no antitumor activity is seen at the initial doses tested, further dose escalation studies are performed. Without being limited by theory, immunotherapy prodrugs with TLR7 and TLR9 agonists should induce and/or enhance an immune response in both a syngeneic renal carcinoma model and in renal carcinoma patients. Broad activation of the innate immune response in tumors themselves should ultimately lead to an adaptive, T-cell mediated immune response not only against mutated proteins, but also against non-mutated but overexpressed tumor associated antigens and possibly endogenous retroviruses.

Small drug conjugates described herein (i.e., prodrugs) are easy to synthesize and have a favorable PK over antibody-drug conjugates. The prodrugs described herein present a versatile chemical platform that allows for the design of multiarm variants for attachment of different functional groups that can be used for (a) simultaneous imaging with a chelating moiety, (b) combination therapy with a beta or alpha emitting radioisotopes, (c) increasing the payload, (d) combining various proinflammatory molecules such as TLR an STING agonists, for example.

### Example 6

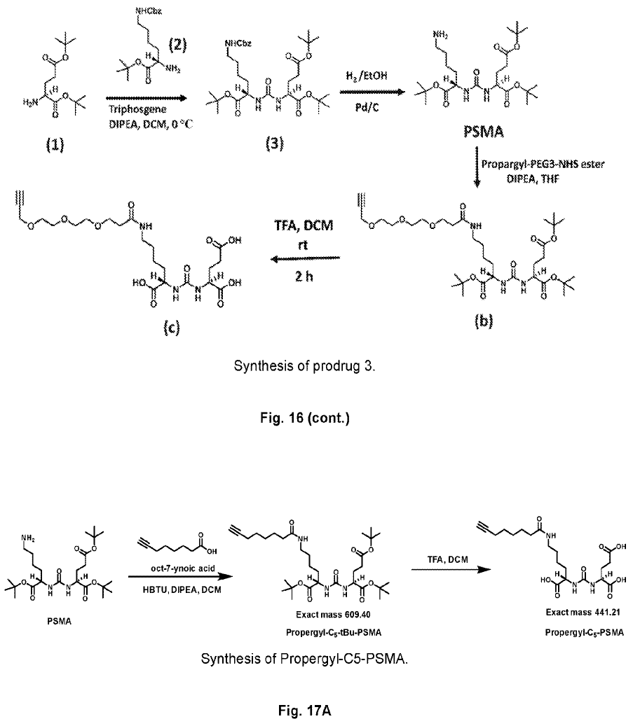

This example describes synthesis of proinflammatory prodrug PSMA-Cathepsin-Gardiquimode (FIG. 16A).

Gardiquimod (16 mg, 0.05 mmol) was diluted in THF/DMF (4 mL/2 mL) and DIPEA (60 mg, 0.46 mmol) was added under nitrogen at 0° C. A solution of Azido-PEG-Val-Cit-PAB-PNP (38 mg, 0.05 mmol) in THF/DMF (4 mL/2 mL) were added drop wise to this solution at 0° C. and stirring was continued for 24 h. Reaction mixture was concentrated and pure prodrug was isolated as a sticky liquid from silica gel column chromatography, 12% methanol in CH₂Cl₂ solvent mixture (14 mg, 30%). MS (ESI) m/z calcd: 947.50; found: 948.52 ([M+H]⁺).

Synthesis of Prodrug 3 (FIG. 16B). In a round-bottom flask triphosgene (2.9 g, 10 mmol) was suspended in DCM (50 mL) and stirred at 0° C. A solution of Cbz-Lys-Ot-Bu. HCl (10 g, 27 mmol) and DIPEA (10.4 mL, 60 mmol) in DCM (50 mL) was added dropwise to the triphosgene solution over 2.5 h. A solution of L-glutamic acid di-tert-butyl ester hydrochloride (7.96 g, 27 mmol) containing DIPEA (10.4 mL, 60 mmol) and DCM (50 mL) was then added in continuous dropwise and allowed to stir for 6 h. The mixture was concentrated to dryness, diluted with 150 mL of ethyl acetate, washed with 2 N NaHSO₄ and brine, and dried over sodium sulfate to yield a yellow oil. Purification by silica gel column chromatography (4:6, EtOAc:Hexane) afforded the desired product as clear oil.

Synthesis of PSMA. Prodrug 3 (621 mg, 1 mmol) was diluted in EtOH (20 mL) and the mixture was degassed for 5 min with nitrogen, followed by addition of 10% Pd on activated charcoal (60 mg), and the mixture was degassed for another 5 min. The mixture was hydrogenated at room temperature with a hydrogen balloon for 36 h and was then filtered and washed with DCM through a Celite pad. The solution was concentrated to provide a syrup which was purified by flash column chromatography, eluting with a 15% gradient of MeOH in CH₂Cl₂, to provide PSMA as a clear colorless syrup. MS (ESI) m/z calcd: 487.33; found: 488.34 ([M+H]⁺).

Synthesis of Prodrug (c). Prodrug (b) (20 mg, 0.03 mmol) were dissolved in 1 ml DCM and stir for 2 min under N₂. To this mixture 1 ml TFA was added and stirring was continued for 2 h. Product formation was confirmed through LC-MS. The final product was obtained by evaporation under vacuum followed by semipreparative HPLC. MS (ESI) m/z calcd: 517.23; found: 518.25 ([M+H]⁺).

Synthesis of PSMA-Cathepsin-Gardiquimode (1) by Click Reaction (FIG. 9). Sodium ascorbate (1.5 mg, 0.072 mmol) and CuSO₄ (0.4 mg, 0.0024 mmol) were dissolved in 0.5 ml water. To this solution a mixture of Prodrug (a) (11 mg, 0.012 mmol) and Prodrug (c) (7 mg, 0.012 mmol) in DCM:MeOH (0.5 ml:0.2 ml) were added drop wise under nitrogen at room temperature. The resulting mixture was then stirred for 24 h. The final product was obtained by evaporation under vacuum followed by HPLC from 90% to 10% solvent-A (1000 ml water and 1 ml TFA) over 20 min following lyophilisation affording 6 mg of PSMA-Cathepsin-Gardiquimode (1). MS (ESI) m/z calcd: 1464.72; found: 1465.76 ([M+H]⁺).

### Example 7

This example describes synthesis of proinflammatory prodrug PSMA-05-Cathepsin-Gardiquimode.

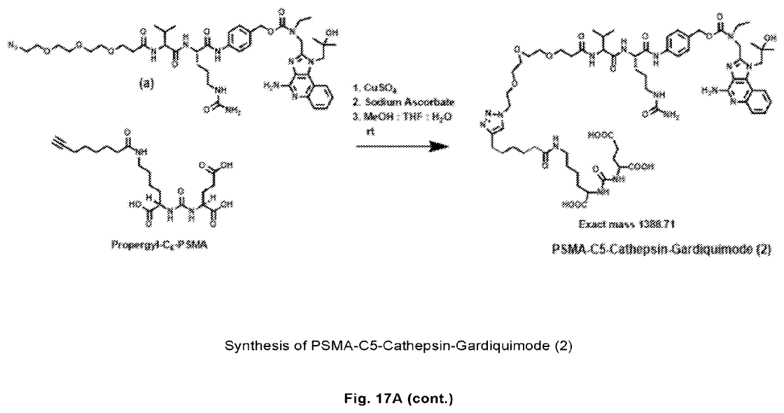

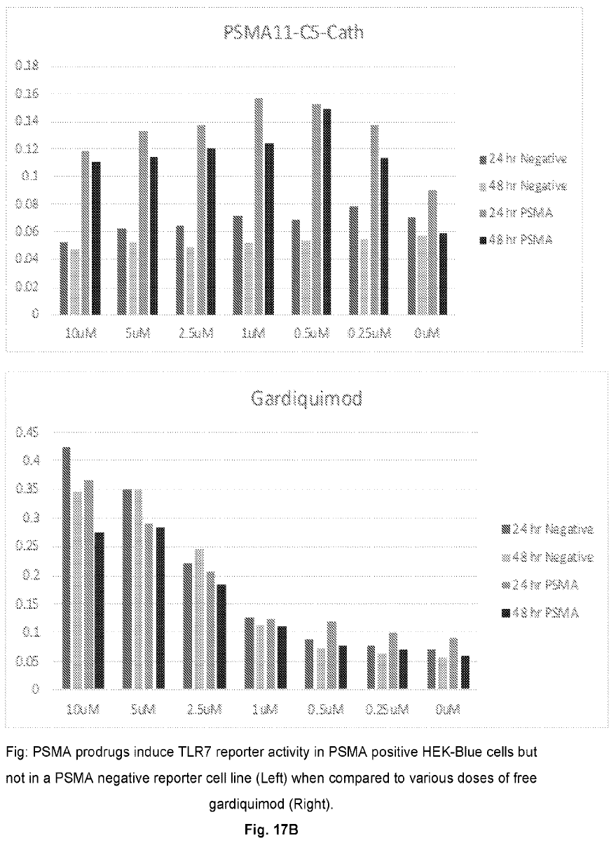

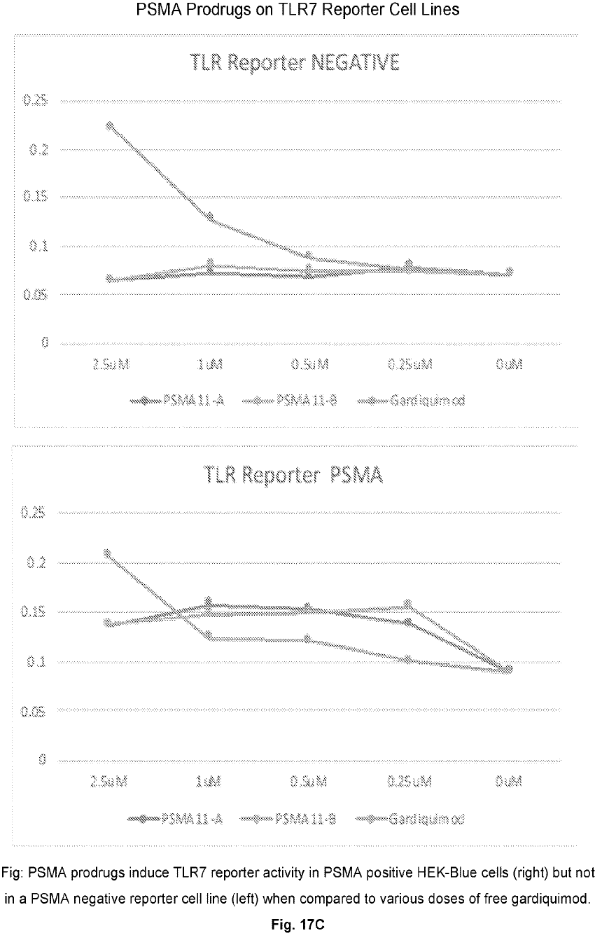

Synthesis of Propergyl-05-PSMA (FIG. 17A). PSMA (121 mg, 0.25 mmol) and oct-7-yonic acid (35 mg, 0.25 mmol) were added in DMF (1 mL) under nitrogen. To this solution HBTU (189 mg, 0.50 mmol) and DIPEA (130 μL, 0.75 mmol) were also added and stirring was continued for 6 h. Reaction mixture was extracted from water and ethyl acetate mixture by collecting the organic layer. The organic layer was evaporated and pure prodrug was isolated from silica gel column chromatography, 2% methanol in CH₂C12 solvent mixture. MS (ESI) m/z calcd: 609.40; found: 610.26 ([M+H]⁺).

Synthesis of Propergyl-05-PSMA. Propergyl-05-tBu-PSMA (18 mg, 0.03 mmol) was dissolved in 1 ml DCM and stir for 2 min under N₂. To this mixture 1 ml TFA was added and stirring was continued for 2 h. Product formation was confirmed through LC-MS. The final product Propergyl-05-PSMA was obtained by evaporation under vacuum followed by semipreparative HPLC. MS (ESI) m/z calcd: 441.21; found: 442.11 ([M+H]⁺).

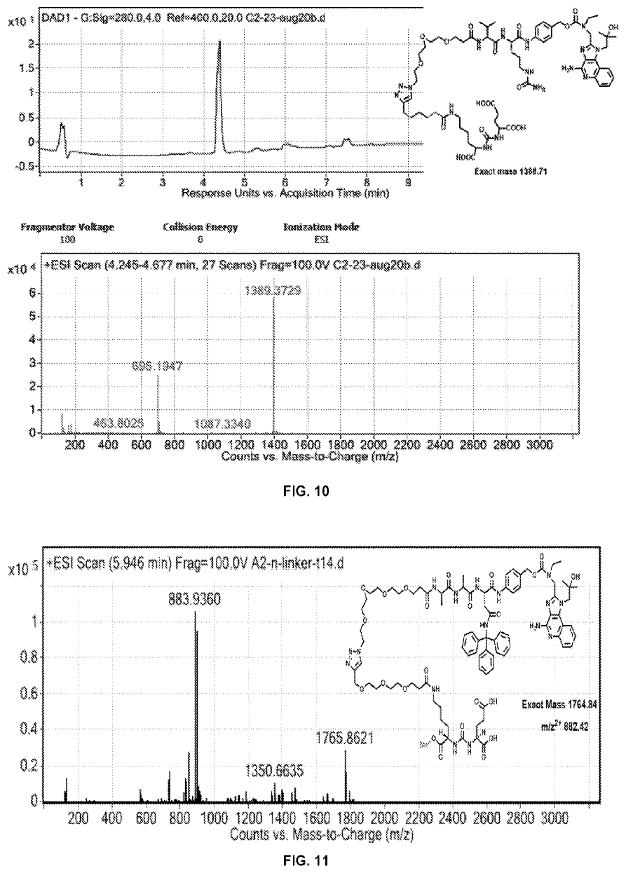

Sodium ascorbate (3 mg, 0.144 mmol) and CuSO₄ (1.2 mg, 0.0072 mmol) were dissolved in 0.5 ml water. To this solution a mixture of prodrug (a) (10 mg, 0.01 mmol) and Propergy-05-PSMA (4.5 mg, 0.01 mmol) in DCM:MeOH (0.5 ml:0.2 ml) were added drop wise under nitrogen at room temperature. The resulting mixture was then stirred for 24 h. The final product was obtained by evaporation under vacuum followed by HPLC from 90% to 10% solvent-A (1000 ml water and 1 ml TFA) over 20 min following lyophilisation affording 4 mg of PSMA-05-Cathepsin-Gardiquimode (2) (FIG. 17A). LC-MS 1389.37 (FIG. 10).

### Example 8

This example describes synthesis of proinflammatory prodrug PSMA-Legu-Gardiquimode.

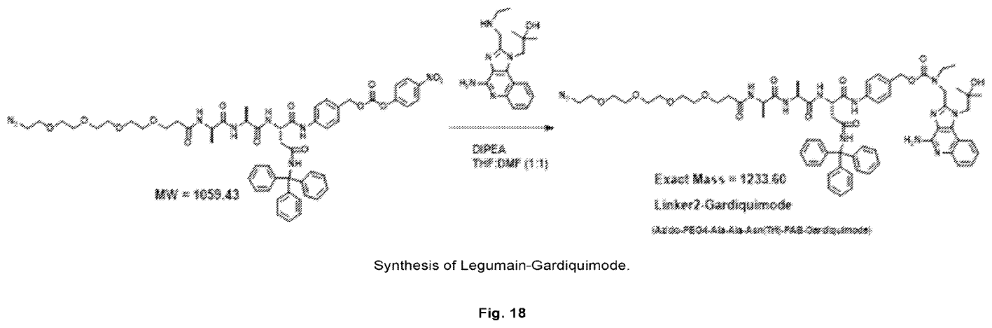

Synthesis of Legumain-Gardiquimode (FIG. 18). Gardiquimod (10 mg, 0.03 mmol) were diluted in THF/DMF (2 mL/1 mL) and DIPEA (60 mg, 0.46 mmol) was added under nitrogen at 0° C. A solution of Azido-PEG-Ala-Ala-Asn(Trt)-PAB-PNP (26 mg, mmol) in THF/DMF (2 mL/1 mL) were added drop wise to this solution at 0° C. and stirring was continued for 24 h. Reaction mixture was concentrated and pure prodrug was isolated as a white powder from HPLC after lyophilisation. MS (ESI) m/z calcd: 1233.60; found: 1234.63 ([M+H]⁺).

Sodium ascorbate (3 mg, 0.144 mmol) and CuSO₄ (1.2 mg, 0.0072 mmol) were dissolved in 0.5 ml water. To this solution a mixture of linker2-Gardiquimode (8 mg, mmol) and prodrug (c) (3.5 mg, 0.007 mmol) in DCM:MeOH (0.5 ml:0.2 ml) were added drop wise under nitrogen at room temperature. The resulting mixture was then stirred for 24 h. The final product was obtained by evaporation under vacuum followed by HPLC from 90% to 10% solvent-A (1000 ml water and 1 ml TFA) over 20 min following lyophilisation affording 5 mg of PSMA-Legu-Gardiquimode (3). LC-MS 1765.86 (FIG. 11).

### Example 9

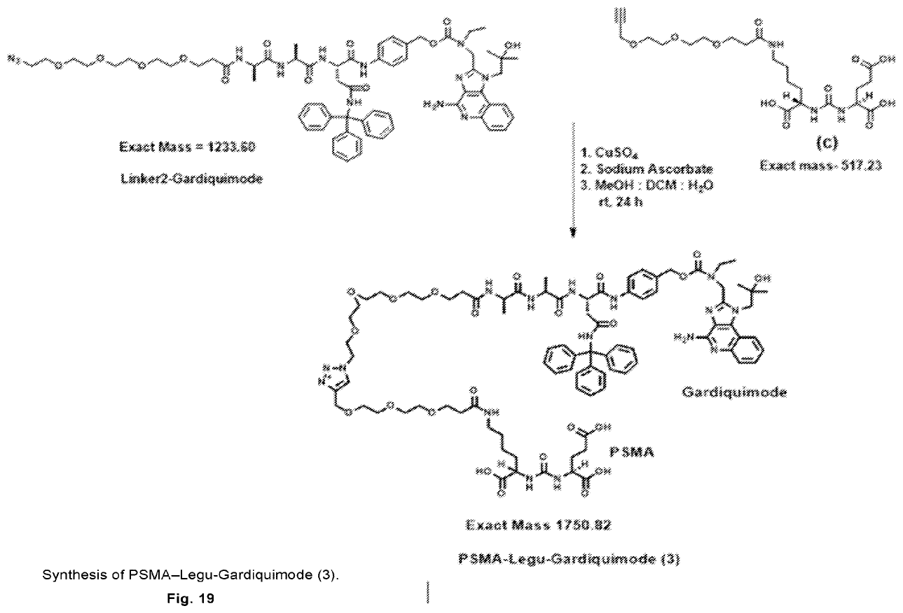

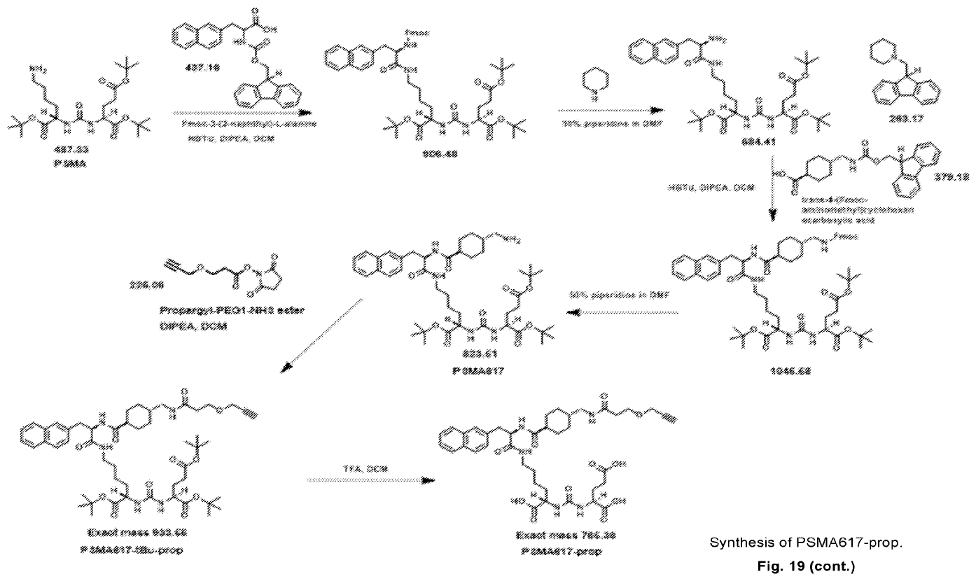

This example describes synthesis of proinflammatory prodrug PSMA617-Legu-Gardiquimode (FIG. 19).

Synthesis of PSMA617-tBu-prop. Synthesis of PSMA617 (exact mass 823.51) has been done via the previously reported procedure. For the synthesis of PSMA617-tBu-prop we treated PSMA617 (82 mg, 0.1 mmol) with Propargyl-PEG1-NHS-ester (22 mg, 0.1 mmol) in presence of DIPEA (55 μL) in DCM (5 ml) under nitrogen at 0° C. reaction was continued for 12 h. Solvent was evaporated and pure prodrug was isolated from HPLC. MS (ESI) m/z calcd: 933.55; found: 934.31 ([M+H]⁺).

Synthesis of PSMA617-prop (FIG. 19). PSMA617-tBu-prop (12 mg, 0.012 mmol) was dissolved in 1 ml DCM and stir for 2 min under N₂. To this mixture 1.2 ml TFA was added and stirring was continued for 3 h. Product formation was confirmed through LC-MS. The final product PSMA617-prop was obtained by evaporation of DCM/TFA mixture under vacuum without any farther purification. MS (ESI) m/z calcd: 765.36; found: 766.16 ([M+H]⁺).

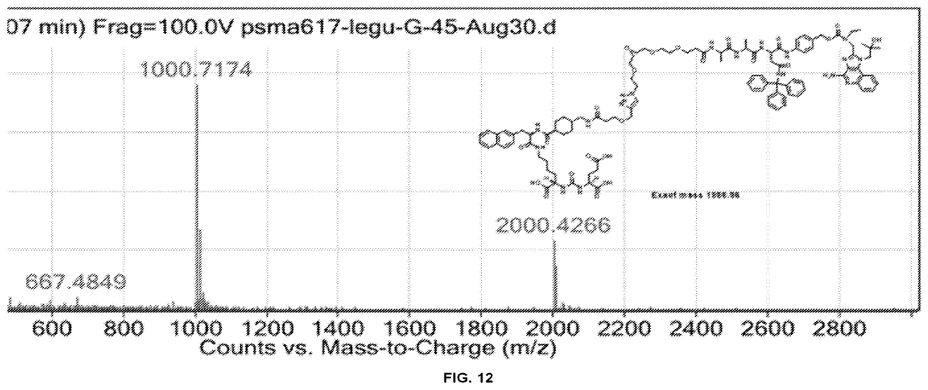

Click reaction for synthesis of final prodrug PSMA617-Legu-Gardiquimode (4). Sodium ascorbate (6 mg, 0.228 mmol) and CuSO₄ (3.6 mg, 0.021 mmol) were dissolved in 0.6 ml water. To this solution a mixture of linker2-Gardiquimode (8 mg, 0.0068 mmol) and PSMA617-prop (5 mg, 0.0065 mmol) in DCM:MeOH (0.5 ml:0.2 ml) were added drop wise under nitrogen at room temperature. The resulting mixture was then stirred for 24 h. The final product was obtained by evaporation under vacuum followed by HPLC from 90% to 5% solvent-A (1000 ml water and 1 ml TFA) over 18 min following lyophilisation affording 3 mg of PSMA617-Legu-Gardiquimode (4). MS (ESI) m/z calcd: 1998.96; found: 2000.42 ([M+H]⁺) (FIG. 12).

### Example 10

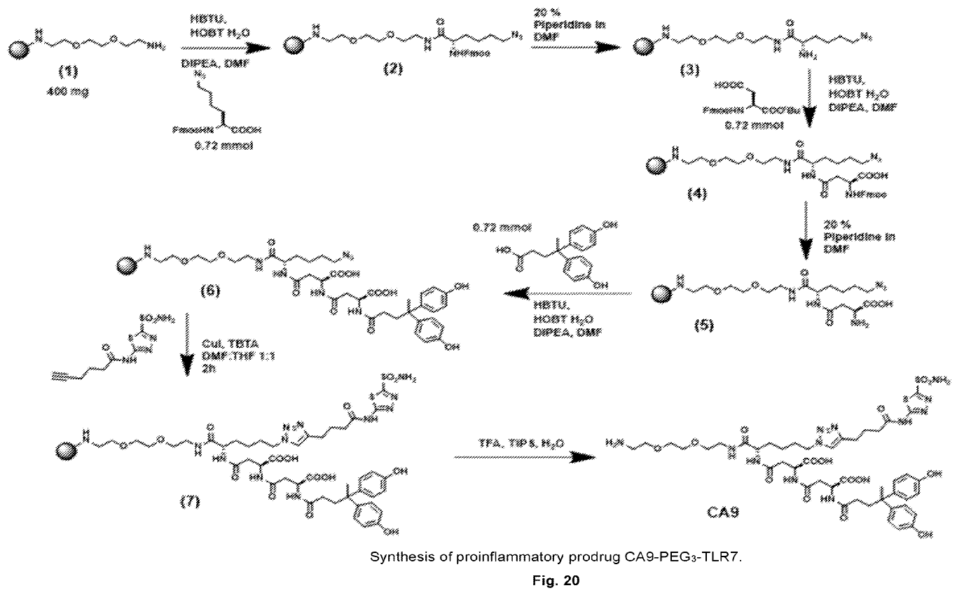

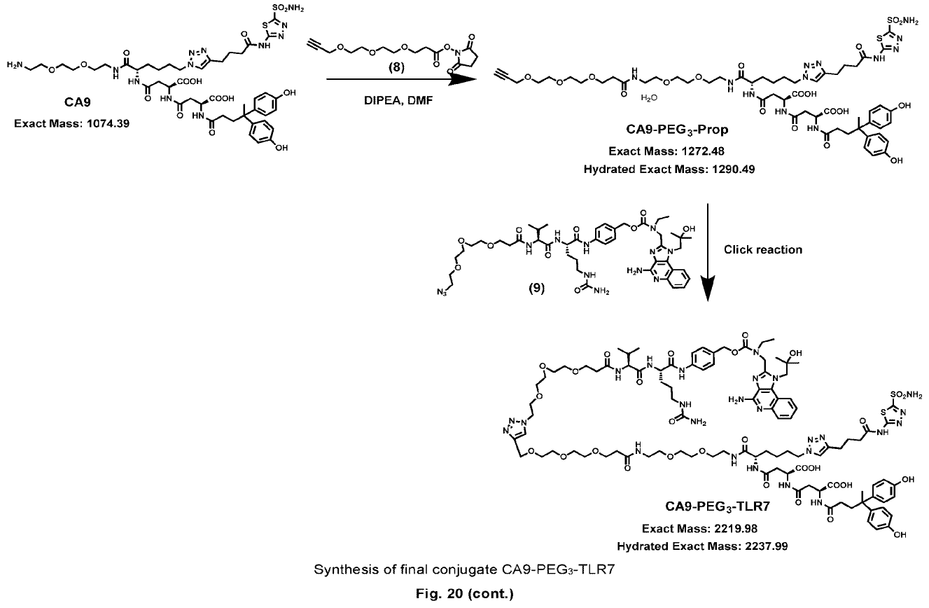

This example describes synthesis of proinflammatory prodrug CA9-PEG₃-TLR7 (FIG. 20).

Synthesis of CA9 ligand. Commercially available pre-loaded 0-bis-(aminoethyl)ethylene glycol on trityl resin (400 mg, 0.24 mmol) was swollen first in DCM (3×5 min×4 ml) and then in DMF (3×5 min×4 ml). Fmoc-protected azidolysine (284 mg, 0.72 mmol), HBTU (274 mg, 0.72 mmol), HOBt·H₂O (110 mg, 0.72 mmol) and DIPEA (238 μl, 1.44 mmol) were dissolved in DMF (4 ml), the mixture was allowed to stand at r.t. for 15 min and then reacted with the resin for 1 h under gentle agitation of N₂. After washing with DMF (6×1 min×4 ml) the Fmoc group was removed with 20% piperidine in DMF (1×2 min×4 ml and 2×10 min×4 ml) and the resin washed with DMF (6×1 min×4 ml) before the peptide was extended 2× with N-α-Fmoc-L-aspartic acid α-tert-butyl ester (296 mg, 0.72 mmol) and 4,4-bis(4-hydroxyphenyl)valeric acid (206 mg, 0.72 mmol) in the indicated order using the same coupling (HBTU/HOBt·H₂O/DIPEA) and Fmoc-deprotection (20% piperidine in DMF) conditions mentioned before. After the last peptide coupling step, a solution of Cul (3.6 mg, 0.02 mmol), TBTA (12.8 mg, 0.02 mmol) and alkyne 11 (198 mg, 0.72 mmol) in a mixture of DMF (2 ml) and THF (2 ml) was prepared and reacted with the resin at r.t. for 2 h. After washing with DMF (6×1 min×4 ml), the prodrug was cleaved by agitating the resin with a mixture of TFA (9 ml), TIPS (500 μl) and H₂O (500 μl) at r.t. for 2 h. The resin was washed with TFA (1×5 min×4 ml) and the combined cleavage and washing solutions added drop-wise to ice cold diethyl ether (100 ml). The precipitate was collected by centrifugation and the product purified by reversed-phase HPLC. After lyophilization CA9 was collected as a white powder (5 mg, 1.7% yield).

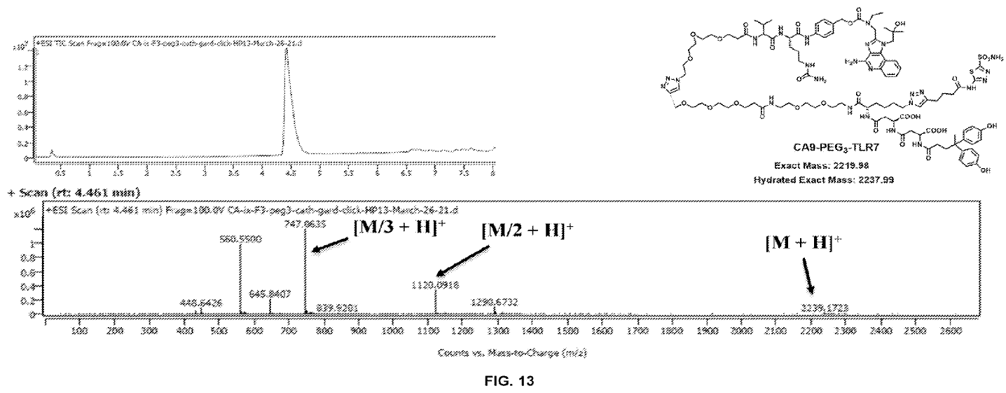

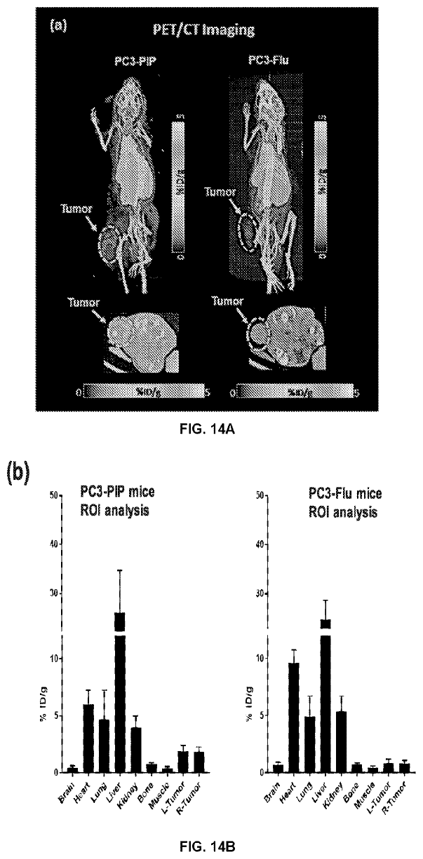

Synthesis of final conjugate CA9-PEG₃-TLR7 (FIG. 20). CA9 was conjugated with the ligand (8) in presence of DIPEA and DMF to produce CA9-PEG₃-Prop. CA9-PEG₃-Prop reacted with prodrug (9) under click condition to produce the final CA9-PEG₃-TLR7 conjugate (FIG. 13).

### Example 11

This example describes synthesis of TLR-LEGU-PSMA-[¹⁹F]SFB (5a) and TLR-LEGU-PSMA-[¹⁸F]SFB (5b).

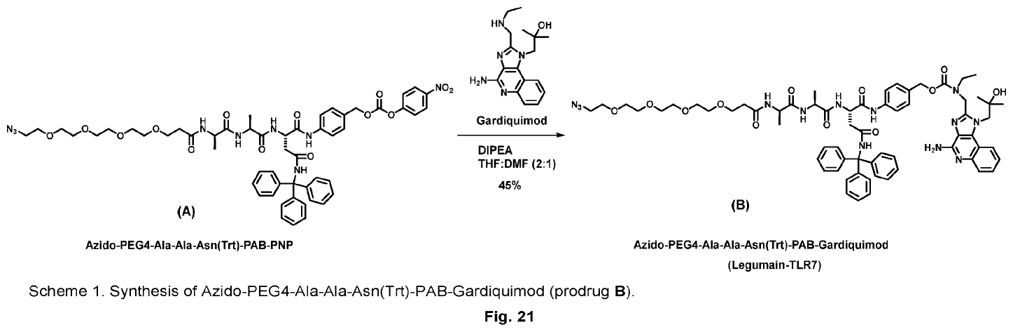

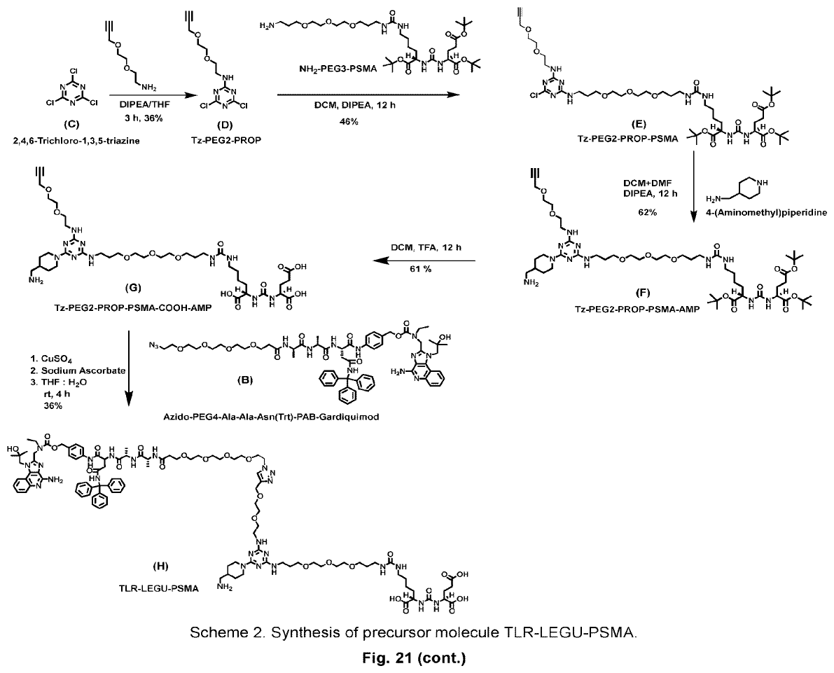

Scheme 1 illustrates the synthesis of prodrug B in presence of DIPEA and THF: DMF mixture with 45% yield (FIG. 21). Scheme 2 shows the synthetic route for key precursor molecule TLR-LEGU-PSMA (H). 2,4,6-Trichloro-1,3,5-triazine has been chosen as a center core to synthesize the tri-modal molecular scaffold. Propargyl-PEG2-amine was conjugated with 2,4,6-Trichloro-1,3,5-triazine in stoichiometric manner for a monosubstituted product; prodrug D. Prodrug D was then conjugated with NH₂-PEG3-PSMA through the second halogen atom of the triazine ring to produce prodrug E with 46% yield. Synthesis of NH₂-PEG3-PSMA (prodrug 4) has been described in scheme 51 (SI) via a three steep synthetic rout. Prodrug E was then conjugated with 4-(Aminomethyl)piperidine through the third halogen atom of the triazine ring to afford prodrug F with 62% yield. Due to the higher reactivity of the secondary amine of 4-(Aminomethyl)piperidine, the primary amine remains available to incorporate the ¹⁸F labeled prosthetic group. The tertiary butyl groups were then removed in presence of TFA to give prodrug G in 61% yield. A click reaction was then performed between prodrug G and prodrug B to produce the precursor molecule TLR-LEGU-PSMA (H), with 36% yield. Then the immunotherapeutic standard drug conjugate TLR-LEGU-PSMA-[¹⁹F]SFB (I) was synthesized by the reaction between precursor molecule (H) and [¹⁹F]SFB with 57% yield.

Radiochemistry. The synthesis of [¹⁸F]SFB was accomplished by a three steep standard method, scheme S2 (SI). (FIG. 21). In brief, trimethylbenzeneaminium triflate undergoes for radiofluorination to produce [¹⁸F]ethyl 4-fluorobenzoate. The ester group was then hydrolyzed in presence of tetrapropylammonium hydroxide (TPAH) to afford [¹⁸F]4-Fluorobenzoic acid. The carboxylic acid group was then activated with O—(N-succinimidyl)-1,1,3,3-tetramethyluronium tetrafluoroborate (TSTU) to produce [¹⁸F]SFB, with 99% of radiochemical purity.

Small Animal PET/CT Imaging (FIG. 14A). The organ uptake and tumor targeting potency of TLR-LEGU-PSMA-[¹⁸F]SFB was evaluated in severe combined immunodeficiency (SCID) mice bearing the PSMA positive and PSMA negative subcutaneous tumor xenografts. FIG. 14A shows the small animal PET/CT imaging studies evaluated by 15 min PET scan followed by 7 min CT scan. The maximum intensity projections (MIP) at 1 h postinjection (p.i.) showed that PSMA positive PC3-PIP tumor was clearly observable while the PSMA negative PC3-Flu tumor has very weak image contrast (similar to muscle uptake).

Quantitative uptake analysis (FIG. 14B) showed that, conjugate TLR-LEGU-PSMA-[¹⁸F]SFB has higher uptake in PC3-PIP (1.9±0.5% ID/g) tumor than in PC3-Flu (0.8±0.3% ID/g) tumors (n=3). At 1 h p.i., the higher level of heart, lung and liver uptake indicates the slow clearance of TLR-LEGU-PSMA-[¹⁸F]SFB from non-specific organs. The observed slow clearance could be due to the low hydrophilic nature of TLR-LEGU-PSMA-[¹⁸F]SFB, which arises due to the presence of hydrophobic gardiquimod molecule and other hydrophobic counterparts.

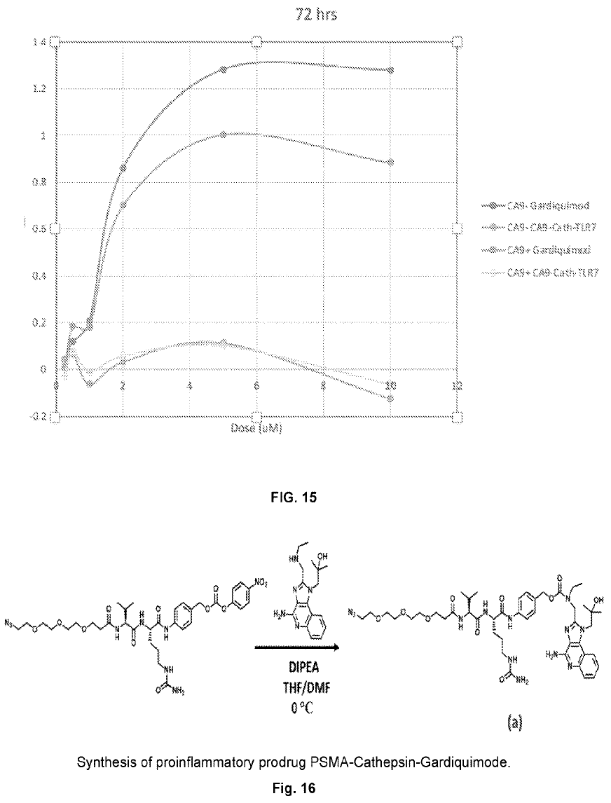

In Vitro Testing of CA9 targeted Prodrug (FIG. 15). The CA9 Cathepsin TLR7 prodrug was tested in CA9 expressing/positive and CA9 negative reporter cells. The Y axis represents optical density. In contrast to the PSMA prodrug data, there was no activation of the TLR pathway, indicating an inability for this prodrug to be endocytosed. While this is negative data for this particular probe, it supports the finding for the PSMA probe as a proof of principle.

What is claimed is:

1. A prodrug comprising:
(i) a targeting moiety that specifically binds to an extracellular antigen;
(ii) a first enzyme-cleavable linker; and
(iii) a first innate immune system activator,
wherein the first linker covalently links the targeting moiety to the innate immune system activator.

2. The prodrug of claim 1, wherein the extracellular antigen is a prostate-specific membrane antigen (PSMA), carbonic anhydrase 9 (CA9), or fibroblast activation protein (FAP).

3. The prodrug of claim 1, wherein the targeting moiety is PSMA-11, PSMA-617, a CA9 small molecule binder, or a FAP inhibitor.

4. The prodrug of claim 3, wherein the FAP inhibitor is FAPI-04.

5. The prodrug of claim 1, wherein the first enzyme-cleavable linker is a cathepsin-cleavable linker or a legumain-cleavable linker.

6. The prodrug of claim 1, wherein the first enzyme-cleavable linker is an azide-polyethylene glycol (PEG)-valine-citrulline-p-aminobenzyl (PAB)-p-nitrophenol (PNP) linker or an alanine-alanine-asparagine legumain linker.

7. The prodrug of claim 1, further comprising a spacer linker between the targeting moiety and the first linker.

8. The prodrug of claim 7, wherein spacer linker is an azide-PEG-maleimide spacer linker or an N-hydroxysuccinimide (NHS)-PEG-Alkyne spacer linker.

9. The prodrug of claim 1, further comprising an albumin binding motif.

10. The prodrug of claim 1, wherein the first innate immune system activator comprises a Toll-like Receptor (TLR) agonist or a Stimulator of Interferon Genes (STING) agonist.

11. The prodrug of claim 10, wherein the TLR agonist is a TLR7 agonist, TLR8 agonist, a TLR9 agonist, or a mixed TLR7/8 agonist.

12. The prodrug of claim 11, wherein the TLR7 agonist is gardiquimod, imiquimod, or telratolimod.

13. The prodrug of claim 11, wherein the mixed TLR7/8 agonist is resiquimod.

14. The prodrug of claim 11, wherein the TLR9 agonist is ODN-2395, CMP-001, or MGN1703.

15. The prodrug of claim 10, wherein the STING agonist is a cyclic dinucleotide.

16. The prodrug of claim 1, further comprising a radiolabel moiety, a second activator of the innate immune system, or a combination thereof.

17. The prodrug of claim 16, wherein the radiolabel moiety is linked to the prodrug by a prosthetic group or a chelator.

18. The prodrug of claim 17, wherein the prosthetic group comprises a benzoate moiety for labeling with a radionuclide selected from ¹⁸F, ¹²³I, ¹²⁴I, ¹³¹I, ⁷⁵Br or ⁷⁶Br linked to the prodrug by a second linker.

19. The prodrug of claim 17, wherein the chelator comprises a 1,4,7,10-tetraazacyclodecane-1,4,7,10-tetraacetic acid (DOTA) derivative for labeling with a radionuclide for imaging selected from ⁶⁷Ga, ⁶⁸Ga, ⁶⁰Cu, ⁶¹Cu, ^{/62}Cu, ⁶⁴Cu, ⁸⁹Zr, ¹⁷⁷Lu, and ^{99m}Tc and/or a radionuclide for radiation therapy selected from ⁶⁷Cu, ¹⁷⁷Lu, ⁹⁰Y, and ²²³Ra.

20. The prodrug of claim 16, wherein the second innate immune activator comprises a proinflammatory molecule linked to the prodrug by a third linker.

21. A prodrug selected from:

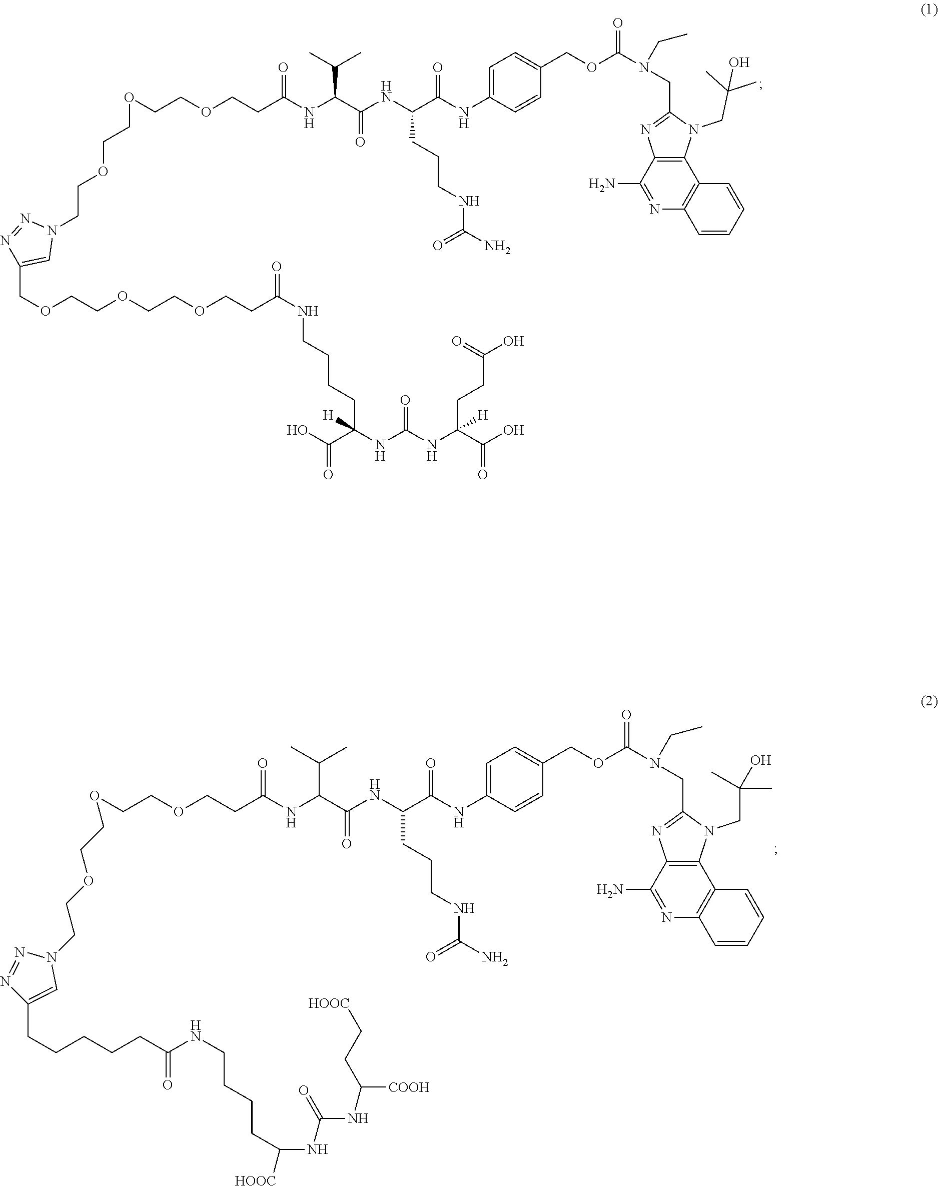 [SMILES: CCN(Cc1nc2c(N)nc3ccccc3c2n1CC(C)(C)O)C(=O)OCc1ccc(NC(=O)[C@H](CCCNC(N)=O)NC(=O)[C@@H](NC(=O)CCOCCOCCOCCn2cc(COCCOCCOCCC(=O)NCCCC[C@H](NC(=O)N[C@@H](CCC(=O)O)C(=O)O)C(=O)O)nn2)C(C)C)cc1 ; CCN(Cc1nc2c(N)nc3ccccc3c2n1CC(C)(C)O)C(=O)OCc1ccc(NC(=O)[C@H](CCCNC(N)=O)NC(=O)C(NC(=O)CCOCCOCCOCCn2cc(CCCCCC(=O)NCCCCC(NC(=O)NC(CCC(=O)O)C(=O)O)C(=O)O)nn2)C(C)C)cc1]

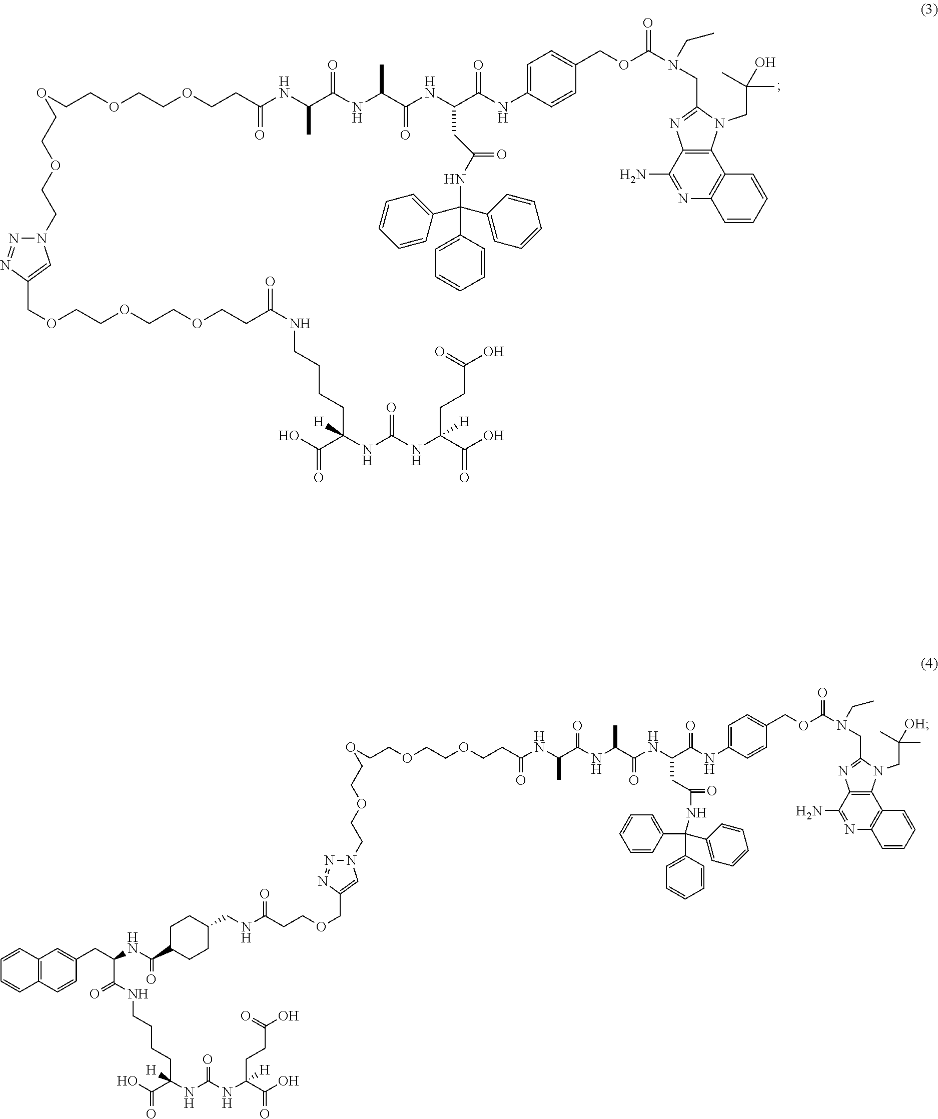

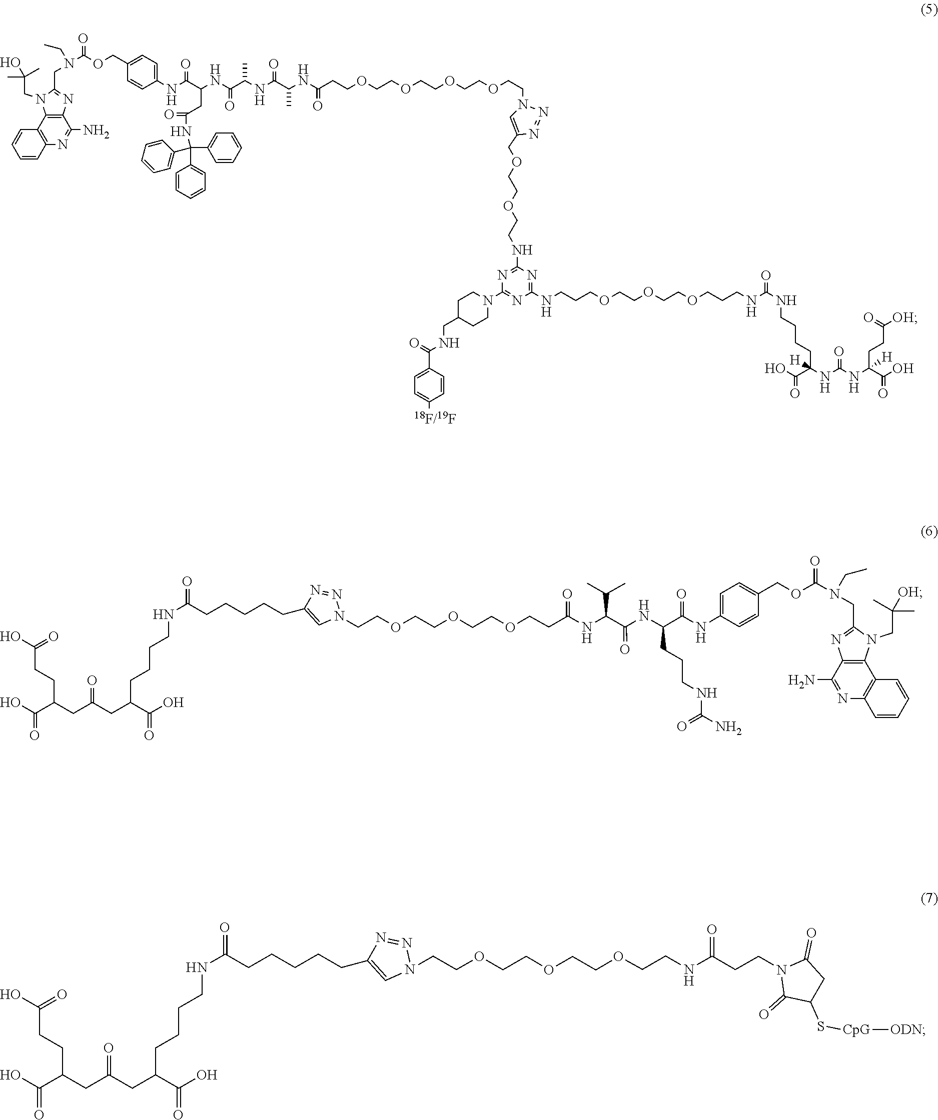

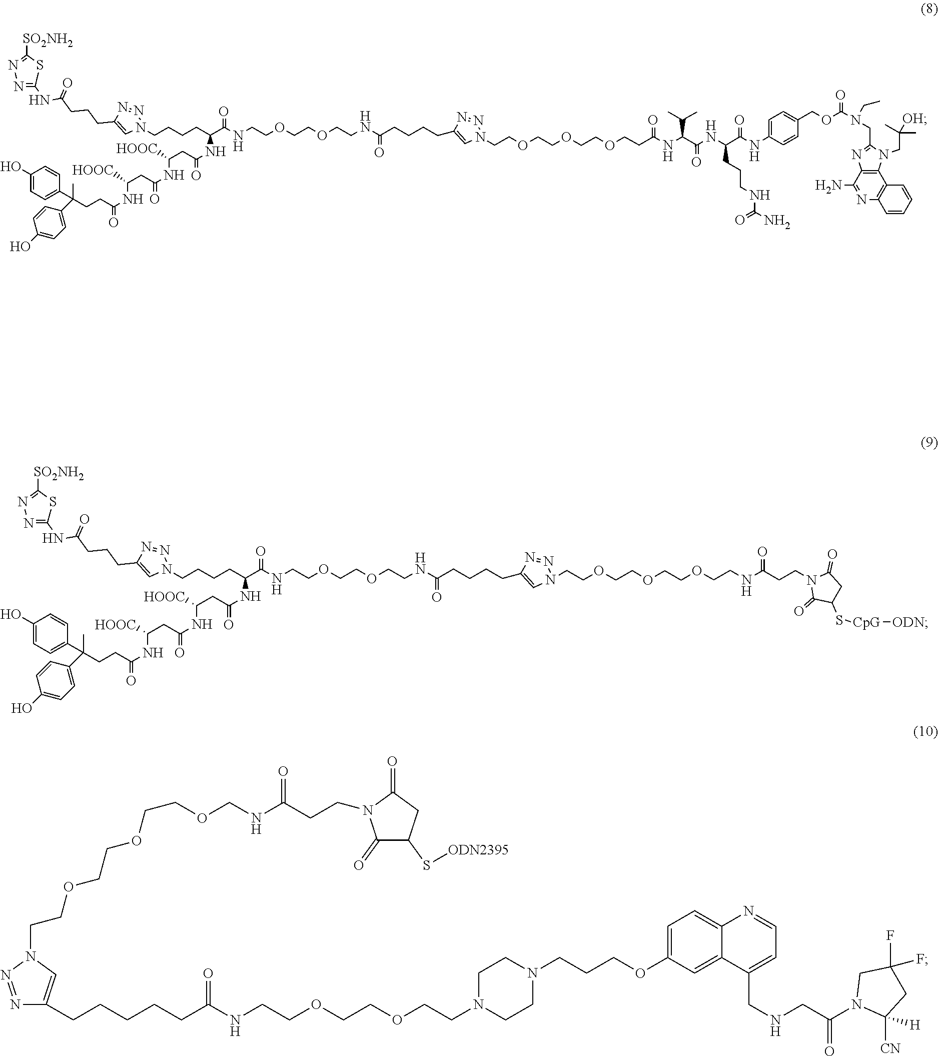

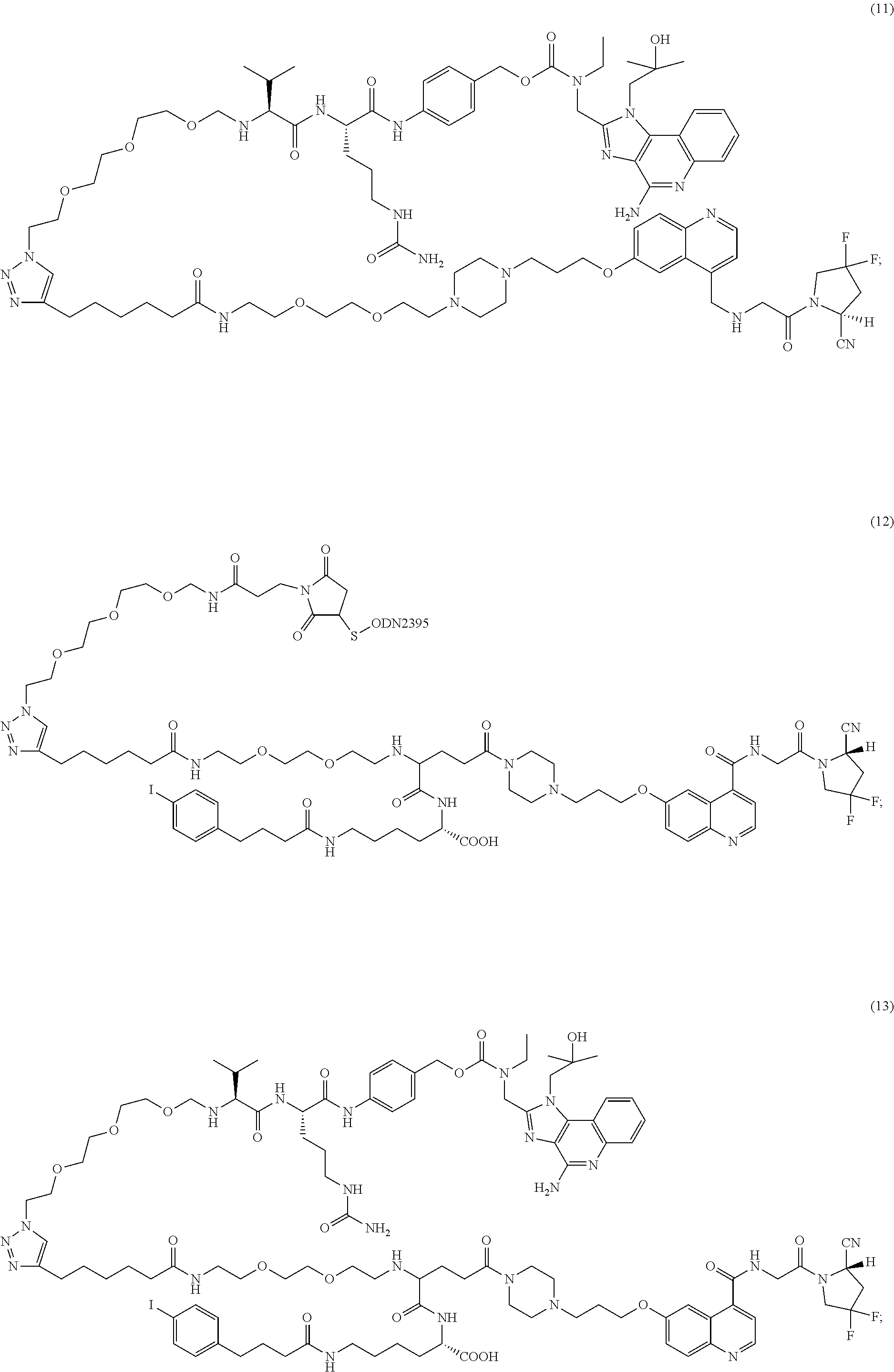

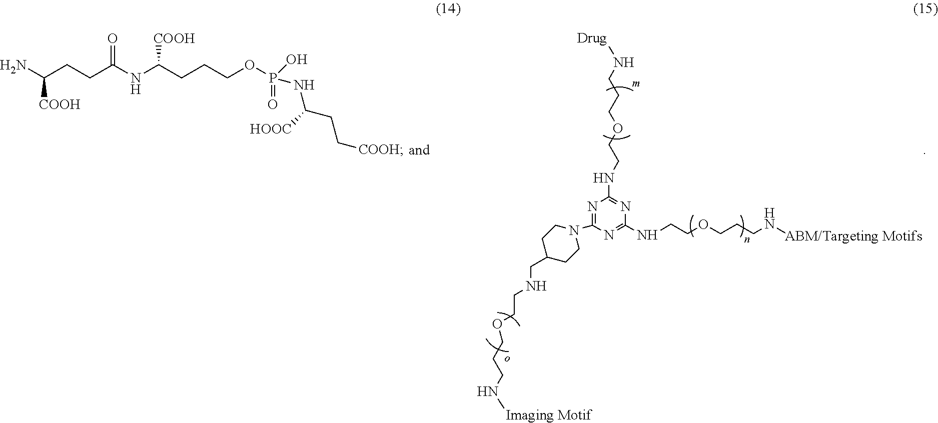

22. A method of treating cancer in a subject comprising: administering to the subject an effective amount of the prodrug of claim 1 or 21, thereby treating the cancer.

23. The method of claim 22, wherein the cancer is kidney cancer, renal cancer, prostate cancer, lung cancer, colon cancer, rectal cancer, urinary bladder cancer, melanoma, oral cavity cancer, pharynx cancer, pancreatic cancer, uterine cancer, thyroid cancer, skin cancer, head and neck cancer, cervical cancer, ovarian cancer, breast cancer, or hematopoietic cancer.

24. The method of claim 22, further comprising administering the prodrug prior to, simultaneously with, or following administration of an immunotherapy.

25. The method of claim 24, wherein the immunotherapy is an interleukin, a cytokine, a chemokine, an immunomodulatory imide drug, CAR-T cells, TCR therapy, a monoclonal antibody, a cancer vaccine, a checkpoint inhibitor, or combinations thereof.

26. A method of imaging cancer in a subject comprising:
(i) administering to the subject a prodrug of any one of claim 16-19;
(ii) optionally administering to the subject immunotherapy after, simultaneously with, or before administration of the prodrug; and
(iii) performing a functional imaging on the subject.

27. A method of monitoring treatment of cancer in a subject comprising:
(i) administering to the subject a prodrug of claim 16;
(ii) optionally administering to the subject immunotherapy after, simultaneously with, or before administration of the prodrug; and
(iii) performing a functional imaging on the subject.

28. The method of claim 26 or 27, wherein the functional imaging is positron emission tomography (PET), single-photon emission computed tomography (SPECT), computed tomography (CT), or any combination thereof.
